(19) United States
(12) Patent Application Publication
(10) Pub. No.: US 2025/0090733 A1
(43) Pub. Date: Mar. 20, 2025

(54) BREAST PUMP DEVICE

(71) Applicant: SHENZHEN LUTE JIACHENG SUPPLY CHAIN MANAGEMENT CO., LTD., Shenzhen (CN)

(72) Inventors: Xue OUYANG, Shenzhen (CN); Wanyuan CHEN, Shenzhen (CN); Guangbao XIE, Shenzhen (CN); Tianlei LI, Shenzhen (CN); Jieqing ZHENG, Shenzhen (CN)

(73) Assignee: SHENZHEN LUTE JIACHENG SUPPLY CHAIN MANAGEMENT CO., LTD., Shenzhen (CN)

(21) Appl. No.: 18/388,372

(22) Filed: Nov. 9, 2023

Related U.S. Application Data
(63) Continuation of application No. PCT/CN2023/119847, filed on Sep. 19, 2023.

(51) Int. Cl. A61M 1/06

(52) U.S. Cl. CPC A61M 1/064; A61M 2205/0216; A61M 2205/07; A61M 2205/123; A61M 2205/125; A61M 2205/3331; A61M 2210/1007

(57) ABSTRACT

A breast pump device is disclosed. The breast pump device includes a main machine housing, a pump coupled to the main machine housing, a breast housing, a milk container, a tee pipe structure detachably attached to the breast housing and the milk container, and a suction diaphragm unit coupled between the pump and the tee pipe structure. The suction diaphragm unit is configured to provide negative pressure to express milk to the milk container via the tee pipe structure and while preventing liquid or moisture from entering the pump.

### CROSS-REFERENCE TO RELATED APPLICATIONS

The present application is a continuation of International Application No. PCT/CN2023/119847, filed on Sep. 19, 2023, the content of which is incorporated herein by reference in its entirety.

### BACKGROUND

The present disclosure relates to a breast pump device, in particular to a portable, wearable, and electrically powered breast pump device.

A portable, wearable, and electrically-powered breast pump device is used for a nursing mother to express milk, who is unable to feed her babies directly due to work or school. Breast pump devices can be hidden underneath the clothing of the nursing mother, thereby ensuring privacy and avoiding unwanted attention and awkwardness while using the breast pump device. Generally, breast pump devices work by creating suction on the breast to release milk from the nursing mother. The released milk is then collected in a container which can be stored in the refrigerator for later use.

### SUMMARY

In one aspect, a breast pump device includes a main machine housing, a pump coupled to the main machine housing, a breast housing, a milk container, a tee pipe structure coupled to the breast housing and the milk container, and a suction diaphragm unit coupled between the pump and the tee pipe structure. The suction diaphragm unit is configured to provide negative pressure to express milk to the milk container via the tee pipe structure while preventing liquid or moisture from entering the pump.

In another aspect, a breast pump device includes a main machine housing, a pump coupled to the main machine housing, a breast housing, a milk container, a suction diaphragm coupled to the breast housing and the milk container, and a filtering cap coupled between the suction diaphragm and the main machine housing.

In still another aspect, a breast pump device includes a main machine housing, a pump coupled to the main machine housing, a breast housing, a milk container, a suction diaphragm detachably attached to the breast housing and the milk container, and a cap coupled to the suction diaphragm and detachably attached to the milk container.

In yet still another aspect, a breast pump device includes a milk container, a breast housing coupled to the milk container via a first end of the milk container, a suction chamber unit coupled to the milk container via a second end of the milk container, and a filtering chamber coupled to the milk container.

### BRIEF DESCRIPTION OF THE DRAWINGS

The accompanying drawings, which are incorporated herein and form a part of the specification, illustrate aspects of the present disclosure and, together with the description, further serve to explain the principles of the present disclosure and to enable a person skilled in the pertinent art to make and use the present disclosure.

FIG. 1A illustrates a side view of an exemplary breast pump device, according to some aspects of the present disclosure.

FIGS. 1B and 1C illustrate side views of partially disassembled breast pump device shown in FIG. 1A, according to some aspects of the present disclosure.

FIG. 1D illustrates a perspective view of the exemplary breast pump device shown in FIG. 1A, according to some aspects of the present disclosure.

FIG. 1E illustrates a bottom view of a main machine of the exemplary breast pump device shown in FIG. 1A, according to some aspects of the present disclosure.

FIG. 1F illustrates a perspective view of the main machine of the exemplary breast pump device shown in FIG. 1A, according to some aspects of the present disclosure.

FIG. 2A illustrates a side view of an exemplary breast pump device, according to some aspects of the present disclosure.

FIG. 2B illustrates an exploded view of the exemplary breast pump device shown in FIG. 2A, according to some aspects of the present disclosure.

FIG. 2C illustrates a front view of the exemplary breast pump device shown in FIG. 2A, according to some aspects of the present disclosure.

FIGS. 2D and 2E illustrate bottom views of a main machine housing of the exemplary breast pump device shown in FIG. 2A, according to some aspects of the present disclosure.

FIGS. 2F and 2G illustrate side views of a filtering cap of the exemplary breast pump device shown in FIG. 2A, according to some aspects of the present disclosure.

FIG. 3A illustrates a side view of an exemplary breast pump device, according to some aspects of the present disclosure.

FIG. 3B illustrates a top view of a milk container and a breast housing of the exemplary breast pump device shown in FIG. 3A, according to some aspects of the present disclosure.

FIG. 3C illustrates an exploded view of the exemplary breast pump device shown in FIG. 3A, according to some aspects of the present disclosure.

FIG. 4A illustrates a front view of an exemplary breast pump device, according to some aspects of the present disclosure.

FIG. 4B illustrates a rear view of the exemplary breast pump device shown in FIG. 4A, according to some aspects of the present disclosure.

FIG. 4C illustrates an exploded view of the exemplary breast pump device shown in FIG. 4A, according to some aspects of the present disclosure.

FIG. 4D illustrates a perspective view of the exemplary breast pump device shown in FIG. 4A, according to some aspects of the present disclosure.

FIG. 4E illustrates a side view of the exemplary breast pump device shown in FIG. 4A, according to some aspects of the present disclosure.

FIG. 4F illustrates another side view of the exemplary breast pump device shown in FIG. 4A, according to some aspects of the present disclosure.

FIG. 4G illustrates a top view of an exemplary breast pump device, according to some aspects of the present disclosure.

FIG. 4H illustrates a cross-section view of the exemplary breast pump device shown in FIG. 4G along an AA plane, according to some aspects of the present disclosure.

FIG. 5A illustrates a top view of an exemplary breast pump device, according to some aspects of the present disclosure.

FIG. 5B illustrates an exploded view of the exemplary breast pump device shown in FIG. 5A, according to some aspects of the present disclosure.

FIG. 5C illustrates a front view of a second housing of the exemplary breast pump device shown in FIG. 5A, according to some aspects of the present disclosure.

FIG. 5D illustrates another front view of the second housing of the exemplary breast pump device shown in FIG. 5A, according to some aspects of the present disclosure.

FIG. 5E illustrates a side view of the second housing of the exemplary breast pump device shown in FIG. 5A, according to some aspects of the present disclosure.

FIG. 5F illustrates a side view of a suction diaphragm unit of the exemplary breast pump device shown in FIG. 5A, according to some aspects of the present disclosure.

FIG. 5G illustrates a side view of a one-way valve of the exemplary breast pump device shown in FIG. 5A, according to some aspects of the present disclosure.

FIG. 5H illustrates a rear view of a first housing of the exemplary breast pump device shown in FIG. 5A, according to some aspects of the present disclosure.

FIG. 5I illustrates a side view of the first housing of the exemplary breast pump device shown in FIG. 5A, according to some aspects of the present disclosure.

FIG. 5J illustrates a front view of the first housing of the exemplary breast pump device shown in FIG. 5A, according to some aspects of the present disclosure.

FIG. 5K illustrates a cross-section view of the exemplary breast pump device shown

in FIG. 5A along a BB plane, according to some aspects of the present disclosure.

FIG. 5L illustrates an enlarged view of the cross-section view shown in FIG. 5K, according to some aspects of the present disclosure.

FIG. 5M illustrates a front view of the first housing without a suction cap and a suction pipe of the exemplary breast pump device shown in FIG. 5A, according to some aspects of the present disclosure.

FIG. 5N illustrates a front view of the suction cap and the suction pipe of the exemplary breast pump device shown in FIG. 5A, according to some aspects of the present disclosure.

FIG. 6 illustrates a block diagram of an exemplary breast pump system, according to some aspects of the present disclosure.

The present disclosure will be described with reference to the accompanying drawings.

### DETAILED DESCRIPTION

Although specific configurations and arrangements are discussed, it should be understood that this is done for illustrative purposes only. A person skilled in the pertinent art will recognize that other configurations and arrangements can be used without departing from the spirit and scope of the present disclosure. It will be apparent to a person skilled in the pertinent art that the present disclosure can also be employed in a variety of other applications.

It is noted that references in the specification to “one embodiment,” “an embodiment,” “an example embodiment,” “some implementations,” etc., indicate that the embodiment described may include a particular feature, structure, or characteristic, but every embodiment may not necessarily include the particular feature, structure, or characteristic. Moreover, such phrases do not necessarily refer to the same embodiment. Further, when a particular feature, structure or characteristic is described in connection with an embodiment, it would be within the knowledge of a person skilled in the pertinent art to affect such feature, structure or characteristic in connection with other implementations whether or not explicitly described.

In general, terminology may be understood at least in part from usage in context. For example, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

It should be readily understood that the meaning of “on,” “above,” and “over” in the present disclosure should be interpreted in the broadest manner such that “on” not only means “directly on” something but also includes the meaning of “on” something with an intermediate feature, a layer, or a structure therebetween, and that “above” or “over” not only means the meaning of “above” or “over” something but can also include the meaning it is “above” or “over” something with no intermediate feature, layer, or structure therebetween (i.e., directly on something).

Further, spatially relative terms, such as “beneath,” “below,” “lower,” “above,” “upper,” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The device may be otherwise oriented (rotated 90 degrees or at other orientations) and the spatially relative descriptors used herein may likewise be interpreted accordingly. In addition, the term “couple”, “coupled to”, or “coupled between” may be understood as not necessarily intended to be “physically joined or attached,” i.e., direct attachment, but can also be interpreted as indirect connection through an intermediate component.

A breast pump device is a medical device or wearable device used to help a nursing mother express her milk. Its general principle is to generate negative pressure through the suction generated by a pump via a suction diaphragm, thereby sucking out the milk. However, in the conventional breast pumps, there are some problems in the installation, fixation, and sealing of the suction diaphragm. For example, due to the improper installation, some unwanted gaps between the suction diaphragm and the air paths may cause spillage or leakage of the liquid (e.g., milk) or moisture, thereby increasing the risk of contamination of the milk. In addition, these unwanted gaps between the suction diaphragm and the air paths may also cause reduction in suction pressure, thereby reducing efficiency and effectiveness of the pump. In another example, the milk container in some of the conventional breast pumps cannot be easily taken apart from the main machine, thereby affecting the use of the product. Furthermore, the suction diaphragm may also be removed from the main machine during the disassembly process or during the process of pouring out milk from the milk container, thereby increasing the chances of additional improper installation each time removing the milk container from the main machine. In addition, although the suction diaphragm may prevent moisture from entering the pump, some of the moisture (e.g., the moisture stays in the breast pump device after washing) may still be sucked into the pump and thus cause the pump to malfunction.

To address one or more of the aforementioned issues, the present disclosure introduces a solution in which a diaphragm cover is detachably attached to the suction chamber and secures the suction diaphragm to the suction chamber of a tee pipe structure or a milk container. The diaphragm cover may limit and pre-press the suction diaphragm before installation, thereby avoiding various improper installation problems. In some implementations, the diaphragm cover may be fastened to the suction diaphragm during installation or pouring out milk from the milk container, thereby reducing the chance of spillage or leakage of the milk or accidental removal of the suction diaphragm. By using the diaphragm cover, the milk pumping effect may also be improved due to the pre-pressing and sealing of the suction diaphragm. In some implementations, different solutions are also introduced to prevent moisture or liquid from being sucked into the pump so as to effectively improve and extend the lifetime of the breast pump device.

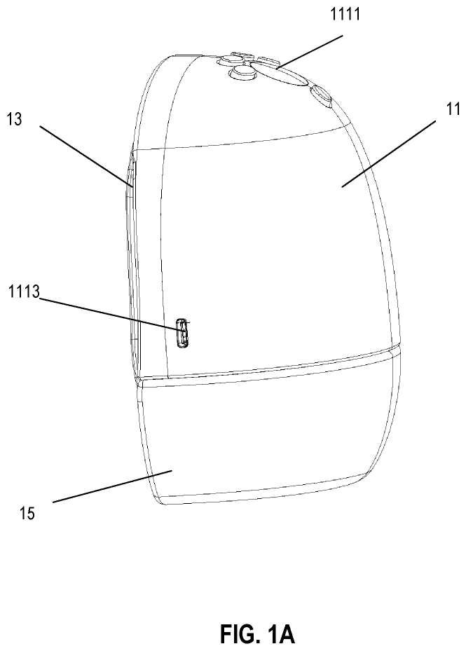

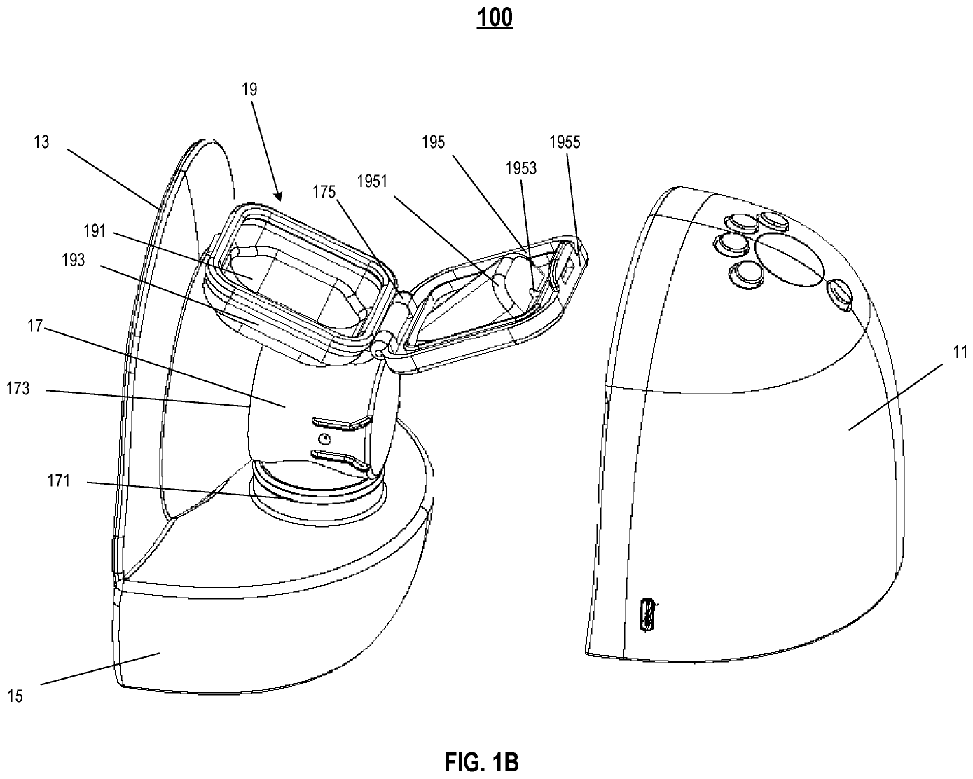

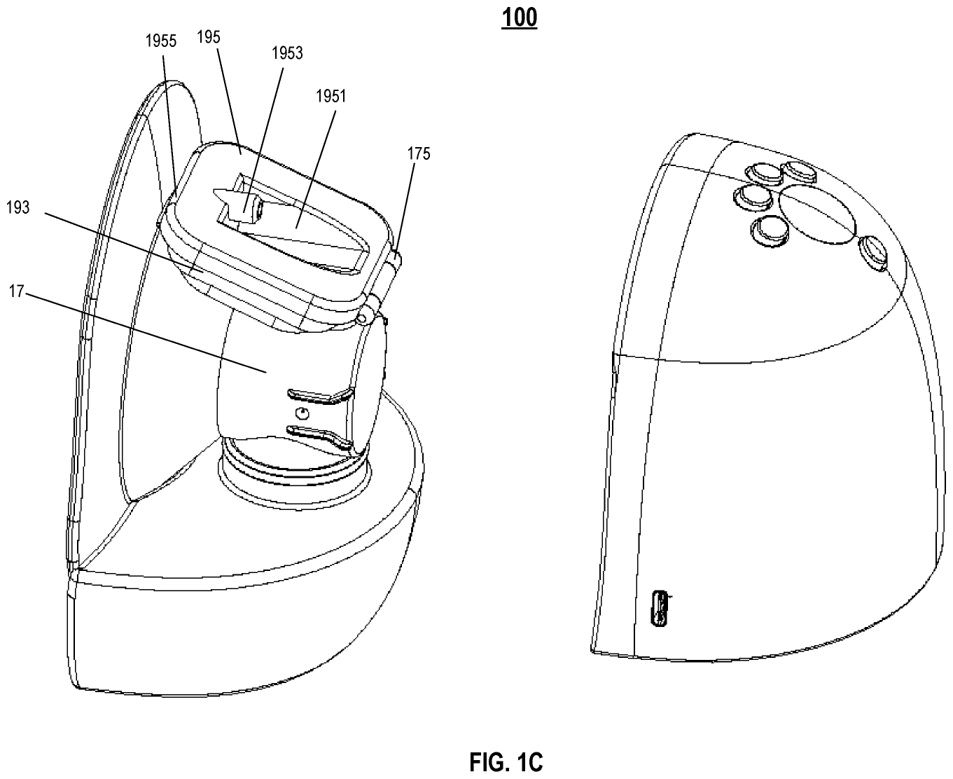

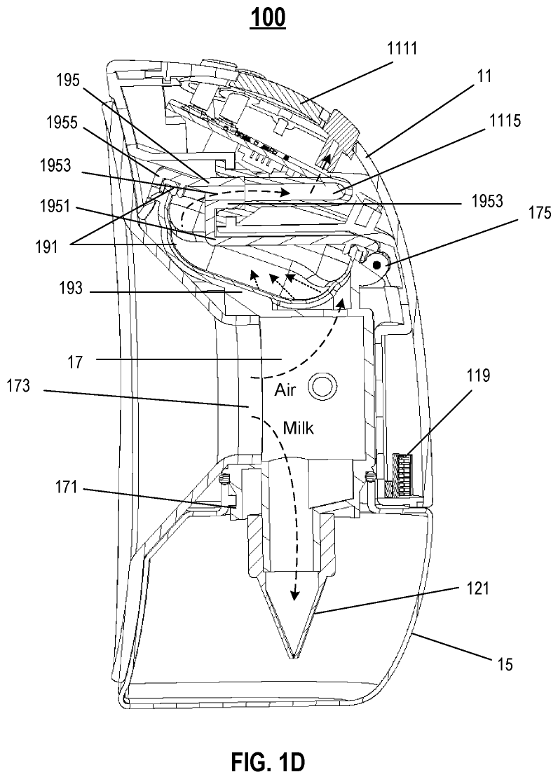

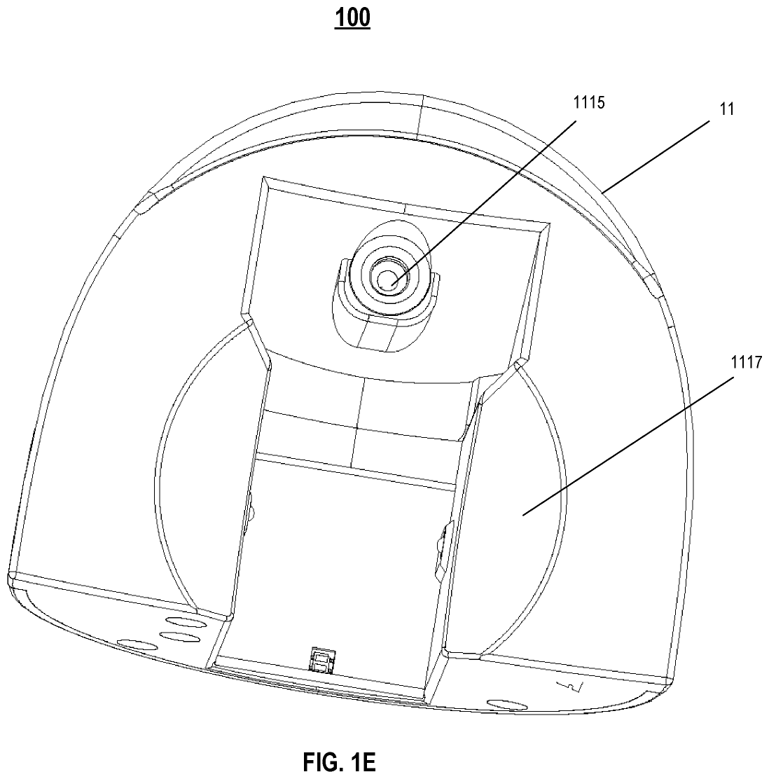

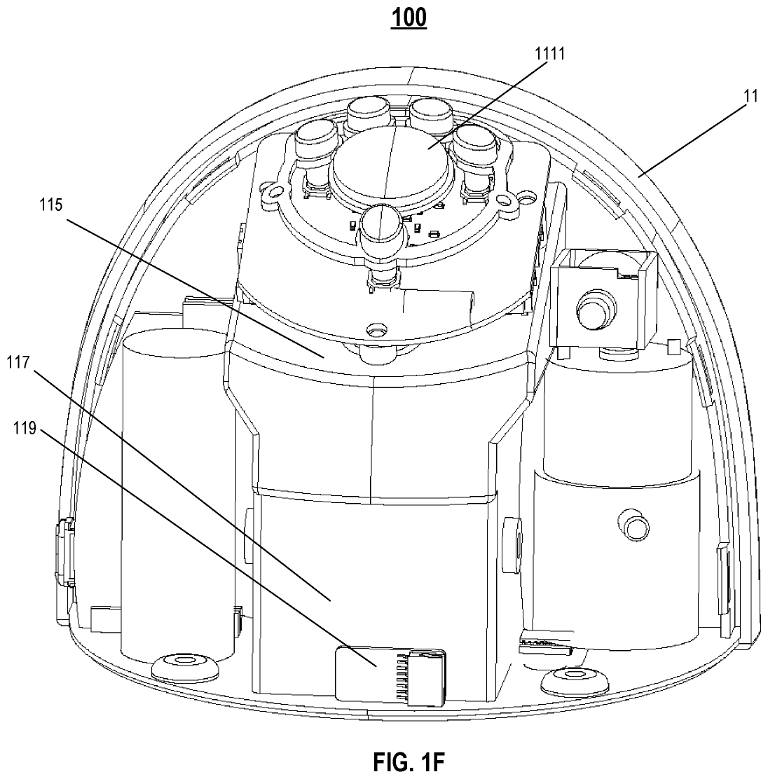

FIG. 1A illustrates a side view of an exemplary breast pump device 100, according to some aspects of the present disclosure. As shown in FIG. 1A, breast pump device 100 includes a main machine housing 11, as well as one or more buttons 1111 disposed on a top portion of main machine housing 11. Breast pump device 100 may be, at least in part, wearable or disposed inside a bra. Buttons 1111 disposed on the top portion of main machine housing 11 may be easily accessed or touched by a user to control the operations of breast pump device 100. In some implementations, buttons 1111 may be disposed in other portions of main machine housing 11. In some implementations, buttons 1111 may include a power button, a switch button, or a control interface on which a user may start, stop, or change a pumping cycle, increase or decrease peak pump pressure, or switch between different pre-programmed or pre-determined pressure profiles (e.g., mimicking a sucking pattern of a baby). Breast pump device 100 may further include a power port 1113 on a side of main machine housing 11. Power port 1113 may charge a power source of the breast pump device or receive power provided from outside beast pump device 100. The power source can be a battery or any other type of portable power source. Main machine housing 11 may accommodate a main machine of breast pump device 100, which includes several components of the main machine to be discussed later.

Breast pump device 100 may further include a breast housing 13 that is detachably attached to main machine housing 11. In some implementations, breast housing 13 can be attached to machine housing 11 by, for example, fastening, buckling, locking, or fitting with each other. When attached together, breast pump device 100 may be leak-proof that prevents water, moisture, and air from entering itself. Breast housing 13 may be separated from main machine housing 11, so that one or both of them can be easily cleaned by user. Breast housing 13 may have a concave surface on a side opposite to main machine housing 11 to accommodate a breast of a nursing mother.

Breast pump device 100 may further include a milk container 15 that is detachably attached to at least one of main machine housing 11 or breast housing 13. In some implementations, milk container 15 can be attached to machine housing 11, breast housing 13, or both by, for example, fastening, buckling, locking, or fitting with each other. When attached together, breast pump device 100 can be leak-proof. When separated, milk container 15 can be easily cleaned by user. In some implementations, milk container 15 may be detachably attached to at least one of main machine housing 11 or breast housing 13 via other components that are coupled between milk container 15 and at least one of main machine housing 11 or breast housing 13. Milk container 15 is configured to collect milk from the breast of the nursing mother after expressing the milk.

FIGS. 1B and 1C illustrate side views of partially disassembled breast pump device 100, according to some aspects of the present disclosure. As shown in FIG. 1B, breast pump device 100 may further include a tee pipe structure 17 detachably attached to breast housing 13, milk container 15, and main machine housing 11. In some implementations, tee pipe structure 17 may include a flange portion 173 that can be fit to a nipple of a breast to collect milk via a path connecting to breast housing 13. An air path portion (not shown) is connected to a suction diaphragm unit 19 to provide suction pressure. A milk portion (not shown) is connected to milk container 15 to guide the milk into milk container 15. In some implementations, tee pipe structure 17 may include a thread structure 171 that can be fastened tightly with milk container 15. In some implementations, flange portion 173 may be independent from tee pipe structure 17, such as a part of breast housing 13. In some implementations, thread structure 171 may be independent from tee pipe structure 17, such as a part of milk container 15.

As shown in FIG. 1B, suction diaphragm unit 19 may include a suction chamber 193, a suction diaphragm 191 detachably attached to suction chamber 193, and a diaphragm cover 195 detachably attached to suction chamber 193 that secures suction diaphragm 191 in a fixed position. In some implementations, at least some of the components in suction diaphragm unit 19 and tee pipe structure 17 are connected via a shaft 175 so that at least one of diaphragm cover 195 or suction diaphragm 191 can be flipped over while still being attached to suction chamber 193 or tee pipe structure 17. In such case, when components of breast pump device 100 are being washed, all the components of suction diaphragm unit 19 may remain attached to tee pipe structure 17 so that no component will be omitted during the disassembly and assembly.

Shaft 175 can be independent from suction diaphragm unit 19 or a part of suction chamber 193. In some implementations, shaft 175 can be coupled to suction chamber 193 or tee pipe structure 17. In some implementations, suction chamber 193 and diaphragm cover 195 may include a rigid material, such as Acrylonitrile Butadiene Styrene (ABS) plastic, polypropylene, polystyrene, nylon, polycarbonate, methacrylate, or a combination of two or more of these materials. Suction diaphragm 191 may include stretchy or elastic material, such as flexible plastic, silicone, rubber, resin, epoxy, or a combination of two or more of these materials. In some implementations, suction chamber 193 and diaphragm cover 195 may be made of a stiffer material than that of suction diaphragm 191. In some implementations, the material used for suction diaphragm 191 is chosen based on its ability to rebound against the high vacuum generated by breast pump device 100. Suction diaphragm 191 may be installed and fit over a rim of suction chamber 193 such that suction chamber becomes airtight sealed. Diaphragm cover 195 may further include a protrusion potion 1951 that fastens suction diaphragm 191 in a secured position. By using suction diaphragm 191, liquid or moisture within milk container 15 or tee pipe structure 17 will be blocked by suction diaphragm 191 so that no moisture or liquid will be sucked into the pump and thus damage the pump.

As shown in FIG. 1C, when diaphragm cover 195 is locked to suction chamber 193 via a buckle 1955, suction diaphragm 191 (shown in FIG. 1B) is fastened between diaphragm cover 195 and suction chamber 193. As such, even with improper installation, suction diaphragm 191 can be secured between diaphragm cover 195 and suction chamber 193. Furthermore, as shown in FIGS. 1B and 1C, diaphragm cover 195 may further include a tube structure 1953 that is connected to an air path to the pump. In some implementations, tube structure 1953 may be inserted into a recessed area of the pump. The air contained by suction diaphragm 191 inside suction chamber 193 and between suction diaphragm 191 and diaphragm cover 195 may be exhausted or vented out to the pump via tube structure 1953, and thus generate negative pressure in suction chamber 193. The negative pressure may express milk out of the breast of a wearer of breast pump device 100, and the milk flows into milk container 15 via tee pipe structure 17.

FIG. 1D illustrates a perspective view of exemplary breast pump device 100, according to some aspects of the present disclosure. As shown in FIG. 1D, a suction hole 1115 of the main machine is coupled to main machine housing 11 and can be connected to tube structure 1953 of diaphragm cover 195. When the nipple of the breast is enclosed by breast housing 13 and aligned with flange portion 173, the pump in the main machine exhausts the air contained by suction diaphragm 191 inside suction chamber 193 via a first side of suction diaphragm 191. When suction diaphragm 191 is deformed by the negative pressure applied on the first side of suction diaphragm 191, air on a second side of suction diaphragm 191 inside suction chamber 193 is exhausted. Since the second side of suction diaphragm 191 is connected to tee pipe structure 17 via an air path (not shown), negative pressure in tee pipe structure 17 is thus also generated. As a result, the nipple enclosed by breast housing 13 and aligned with flange portion 173 of tee pipe structure 17 may express milk, which flows into milk container 15 via a duckbill valve 121. Duckbill valve 121 can allow the milk to be released in one direction (i.e., from tee pipe structure 17 into milk container 15), but not in the opposite direction. It is noted that the duckbill valve disclosed in the present application is not limited to duckbill valve as illustrated, it can be any other types of valves as long as the valve can act as a backflow prevention valve or a one-way valve. The valve may have an elastomeric lip in the shape of a duckbill or any other shape which prevents backflow and allows forward flow.

In some implementations, the pump disclosed in the present application may have a pre-programmed or pre-determined pumping cycle, peak pumping pressure, or different pre-programmed or pre-determined pressure profiles (e.g., mimicking a sucking pattern of a baby). In such case, suction diaphragm 191 is also deformed and recovered according to the pre-programmed or pre-determined pumping cycle, the peak pumping pressure, or the different pre-programmed or pre-determined pressure profiles. It is noted that the pump disclosed hereinafter includes an air pump.

In some implementations, protrusion portion 1951 may include at least one of concave or convex structures, strip structures, protrusion structures, formed on an inner surface of protrusion portion 1951 and surrounding the opening of tube structure 1953. These structures formed on the inner surface of protrusion portion 1951 may retain some liquid (e.g., water, milk, etc.) or moisture inside of the breast pump machine (e.g., within suction diaphragm 191, suction chamber 193, or other components), thereby preventing the liquid or moisture from being sucked into the pump and thus damaging the pump. It is noted that the protrusion portion is only one of the examples that can retain the liquid or moisture inside the breast pump device, it is not limited to the implementations of the present application and can be other structures that provides same or similar technical benefit.

FIG. 1E illustrates a bottom view of exemplary breast pump device 100, according to some aspects of the present disclosure. As shown in FIG. 1E, suction hole 1115 of the main machine is coupled to main machine housing 11 such that it enables the pump to vent the air contained by suction diaphragm 191 inside suction chamber 193. In addition, an inner surface 1117 of main machine housing 11 also has a rounded shape to fit the shape of breast housing 13 during the installation.

FIG. 1F illustrates a perspective view of exemplary breast pump device 100, according to some aspects of the present disclosure. As shown in FIG. 1F, the main machine includes one or more pumps 115, a power system 117 (including a power source, such as a battery), and a control circuit 119 coupled to main machine housing 11. Power system 117 is coupled to pump 115, control circuit 119, or both, and is connected to power port 1113 (shown in FIG. 1A) to charge the breast pump device's battery or receive power from power port 1113. Control circuit 119 is coupled to pump 115 to control the operations of pump 115. For example, control circuit 119 may control a pre-programmed or pre-determined pumping cycle, peak pumping pressure, or different pre-programmed or pre-determined pressure profiles (e.g., mimicking a sucking pattern of a baby) of pump 115. Control circuit 119 may also be coupled to button 1111 such that a user may manually input the instructions, or control the pre-programmed or pre-determined pumping cycle, the peak pumping pressure, or the different pre-programmed or pre-determined pressure profiles via button 1111. In some implementations, pump 115 may further include or be coupled to a solenoid valve (not shown). For example, in an example of the solenoid valve that includes two channels (e.g., an input and an output) and a gate to control the open or close between the two channels, pump 115 may always provide negative pressure while the solenoid valve may open or close the gate between the two channels to allow the negative pressure to pass through. That is, the solenoid valve may be used to control the cycles or profiles of the pumping. In some implementations, the solenoid valve may also be controlled by control circuit 119.

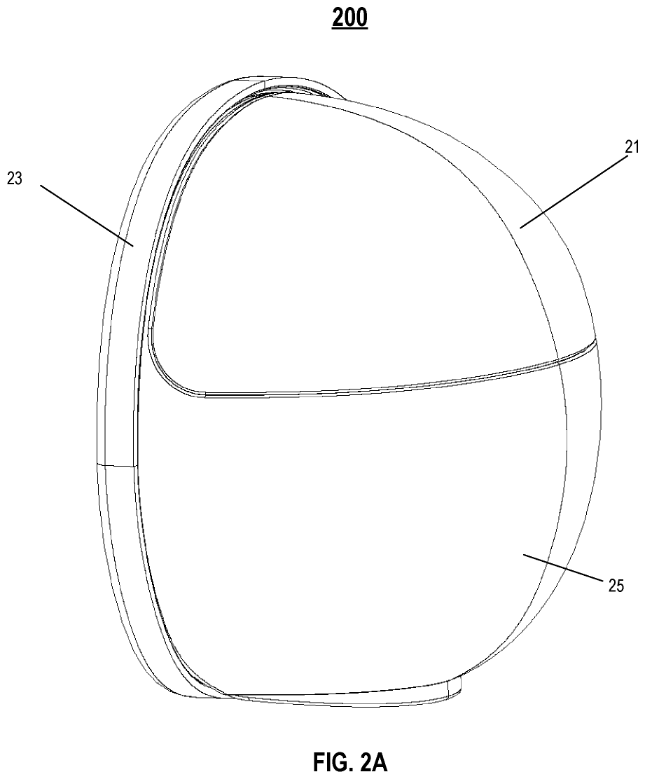

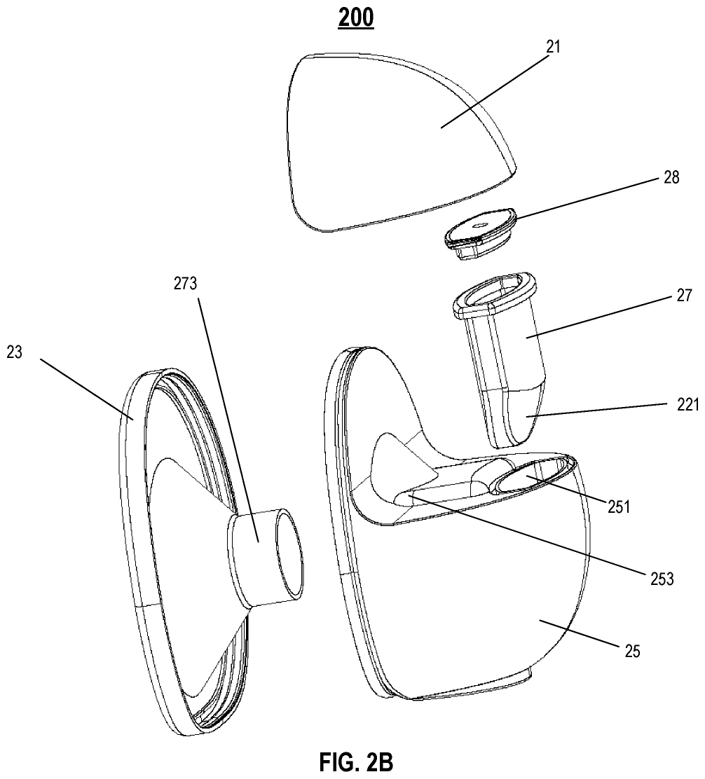

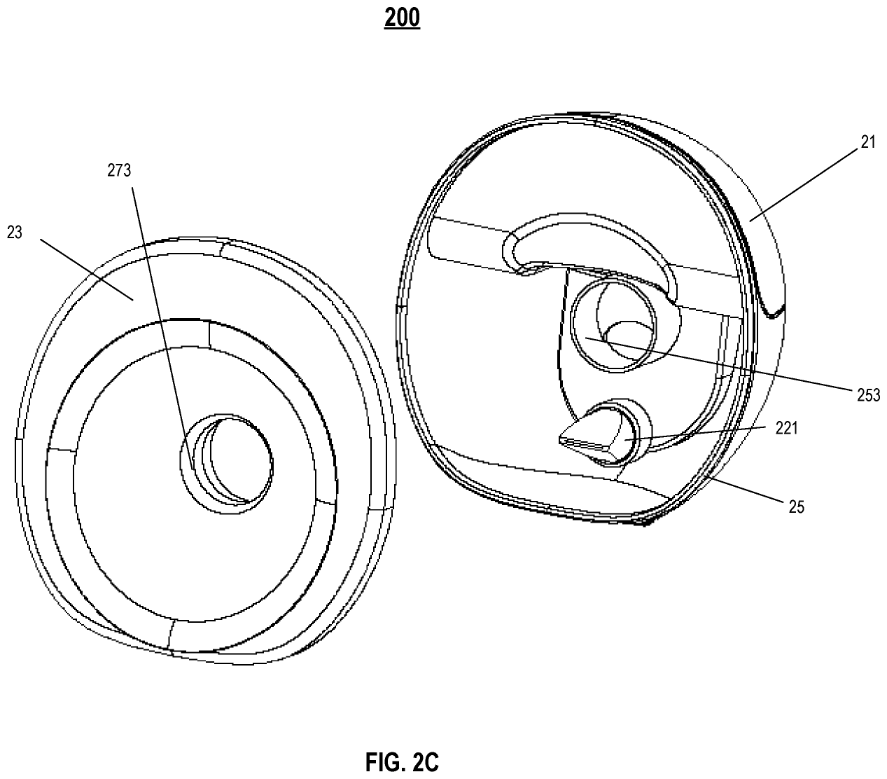

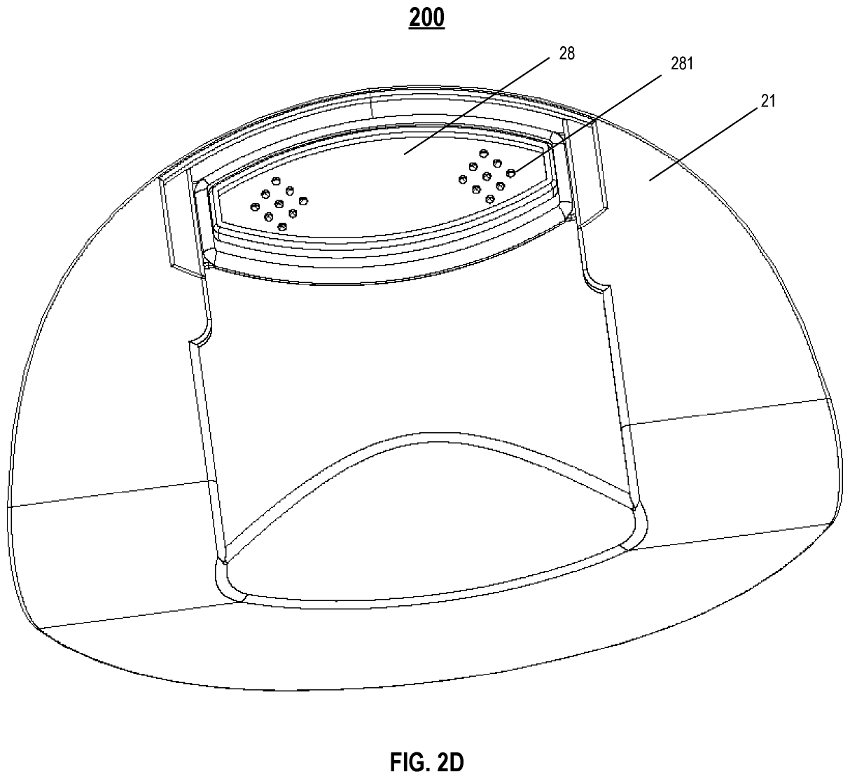

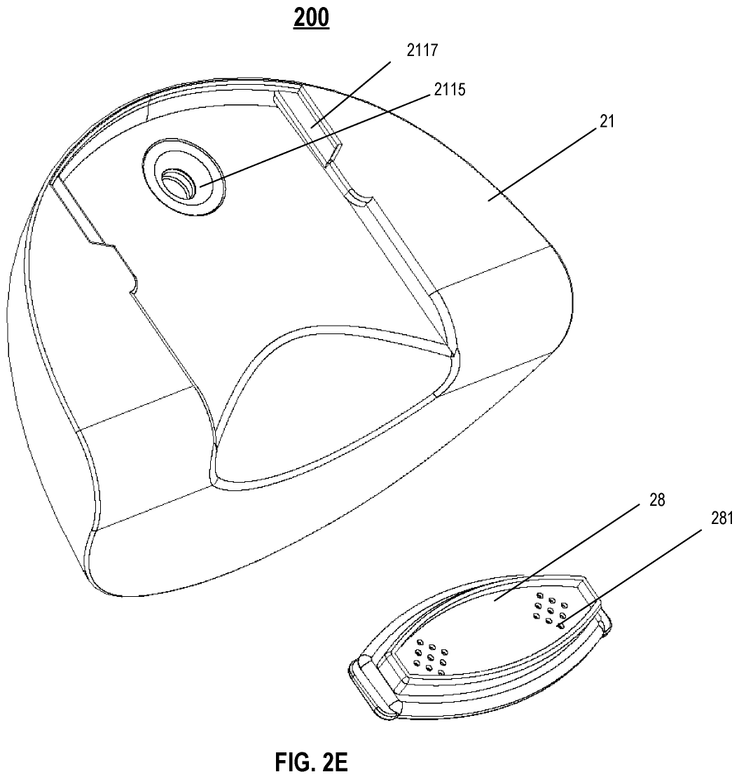

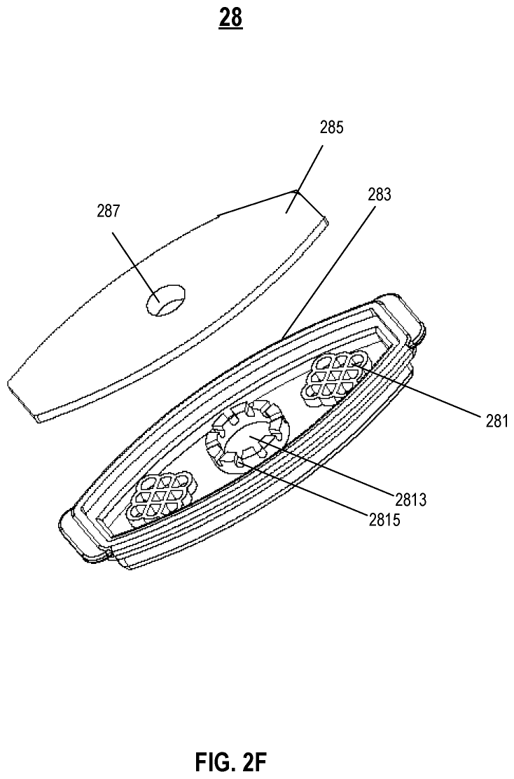

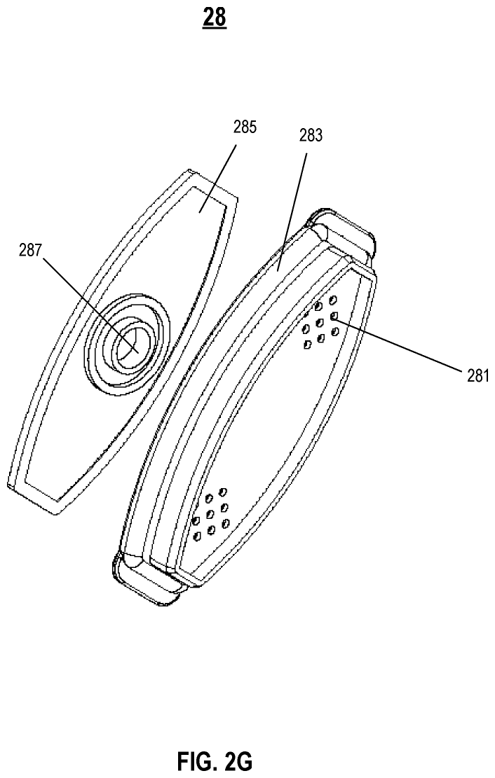

FIG. 2A illustrates a side view of an exemplary breast pump device 200, according to some aspects of the present disclosure. As shown in FIG. 2A, breast pump device 200 includes a main machine housing 21, as well as one or more buttons (not shown) capable of being disposed on a top portion of main machine housing 21. Breast pump device 200 may be, at least in part, wearable or capable of being disposed inside a bra. Main machine housing 21 may further include a power port (not shown) that allows charging of a power source of the breast pump device or receiving of power provided from outside breast pump device 200 via the power port. Main machine housing 21 may accommodate a main machine of breast pump device 200, which includes several components of the main machine. The components may be the same as or similar to those discussed in conjunction with breast pump device 100 shown in FIGS. 1A to 1F.

Breast pump device 200 may further include a breast housing 23 that is detachably attached to main machine housing 21. In some implementations, breast housing 23 can be attached to machine housing 21 by, for example, fastening, buckling, locking, or fitting with each other. When attached together, breast pump device 200 may be leak-proof that prevents water, moisture, and air from entering itself. Breast housing 23 may be separated from main machine housing 21, so that one or both of them can be easily cleaned by user. Breast housing 23 may have a concave surface on a side opposite to main machine housing 21 to accommodate a breast of a nursing mother.

Breast pump device 200 may further include a milk container 25 that is detachably attached to at least one of main machine housing 21 or breast housing 23. In some implementations, milk container 25 can be attached to machine housing 21, breast housing 23, or both by, for example, fastening, buckling, locking, or fitting with each other. When attached together, breast pump device 200 can be leak-proof. When separated, milk container 25 can be easily cleaned by user. In some implementations, milk container 25 may be detachably attached to at least one of main machine housing 21 or breast housing 23 via other components that are coupled between milk container 25 and at least one of main machine housing 21 or breast housing 23. Milk container 25 is configured to collect milk from the breast of the nursing mother after expressing the milk.

FIG. 2B illustrates an exploded view of exemplary breast pump device 200, according to some aspects of the present disclosure. As shown in FIG. 2B, breast pump device 200 may further include a suction diaphragm 27 detachably attached to milk container 25. In some implementations, milk container 25 may include a hole portion 251 in which suction diaphragm 27 can be inserted. Suction diaphragm 27 may include a duckbill valve 221 to guide the milk into milk container 25. Duckbill valve 221 can allow the milk to be released in one direction (i.e., from suction diaphragm 27 into milk container 25), but not in the opposite direction. In some implementations, breast housing 23 may include a flange portion 273 that can be fit to a nipple of a breast to collect milk via a path connecting to breast housing 23. Flange portion 273 may be connected to milk container 25 and suction diaphragm 27 via a through path 253. In some implementations, a filtering cap 28 can be further coupled to a top portion of suction diaphragm 27 or milk container 25. In some implementations, filtering cap 28 is detachably attached to suction diaphragm 27. Filtering cap 28 is also detachably attached to main machine housing 21. As mentioned above, after cleaning or washing the breast pump device, some liquid (e.g., water, milk, etc.) or moisture may be retained inside of the breast pump machine (e.g., within suction diaphragm 27, hole portion 251, through path 253, or other components). Such liquid or moisture will damage the pump or render the pump malfunctioning. By using filtering cap 28, the liquid or moisture inside milk container 25 or suction diaphragm 27 will be blocked by filtering cap 28 so that it would not be sucked into the pump and thus damage the pump. When the nipple of the breast is enclosed by breast housing 23 and aligned with flange portion 273, the pump in the main machine exhausts the air out of suction diaphragm 27 via filtering cap 28 to generate negative pressure. As a result, the nipple enclosed by breast housing 23 and aligned with flange portion 273 of suction diaphragm 27 may express milk, which flows into milk container 25 via a duckbill valve 221. Duckbill valve 221 can allow the milk to be released in one direction (i.e., from suction diaphragm 27 into milk container 25), but not in the opposite direction. It is noted that filtering cap 28 presented in this implementation is only for illustrations, it is not limited to the present implementations and can also be provided or combined in other implementations as well. For example, it can be installed at any position between the tee pipe structure (e.g., suction diaphragm 27 in FIG. 2B) and an air path to the pump (e.g., suction hole 2115 in FIG. 2E), or between the air path to the pump and the pump to further block leakage of liquid or moisture. In some implementations, suction hole 2115 is in a recessed portion of the pump.

FIG. 2C illustrates a front view of exemplary breast pump device 200, according to some aspects of the present disclosure. As shown in FIG. 2C, flange portion 273 of breast housing 23 is coupled to milk container 25 via through path 253 of milk container 25.

FIGS. 2D and 2E illustrate bottom views of a main machine housing 21 of exemplary breast pump device 200, according to some aspects of the present disclosure. As shown in FIGS. 2D and 2E, filtering cap 28 is detachably attached to main machine housing 21 via one or more strip structures 2117 that fasten filtering cap 28 securely to main machine housing 21. In some implementations, filtering cap 28 includes one or more cap openings 281. These cap openings 281 are small and narrow and serve to block or reduce liquid or moisture from passing through while allowing air to pass through. In some implementations, these cap openings 281 may not be aligned with a suction hole 2115 of main machine housing 21 so that the liquid or moisture that passes through will be retained in filtering cap 28 or between filtering cap 28 and suction hole 2115. This may further block or reduce the liquid or moisture from leaking to the pump.

FIGS. 2F and 2G illustrate side views of a filtering cap of exemplary breast pump device 200, according to some aspects of the present disclosure. As shown in FIGS. 2F and 2G, in some implementations, filtering cap 28 may further include a first lid 285 detachably attached to cap body 283. First lid 285 may include a lid opening 287 that is aligned with suction hole 2115 of the main machine as well as cap hole 2813 on cap body 283. In some implementations, lid opening 287 can be replaced by a tube structure such that the tube structure can be inserted into suction hole 2115 of the main machine (e.g., a recessed portion of the pump). In some implementations, one or more concave or convex structures 2815 are coupled to cap body 283 and around cap hole 2813 so as to retain the liquid or moisture in filtering cap 28 (e.g., between cap body 283 and first lid 285). In some implementations, cap openings 281 may be arranged in one or more arrays. By creating more contact surfaces or twisted air path through the entire air path, more liquid or moisture will be retained within filtering cap 28.

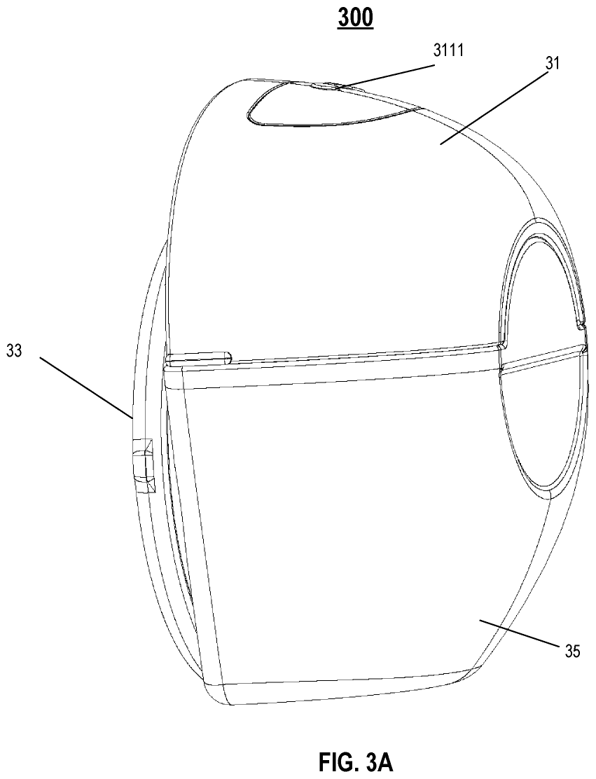

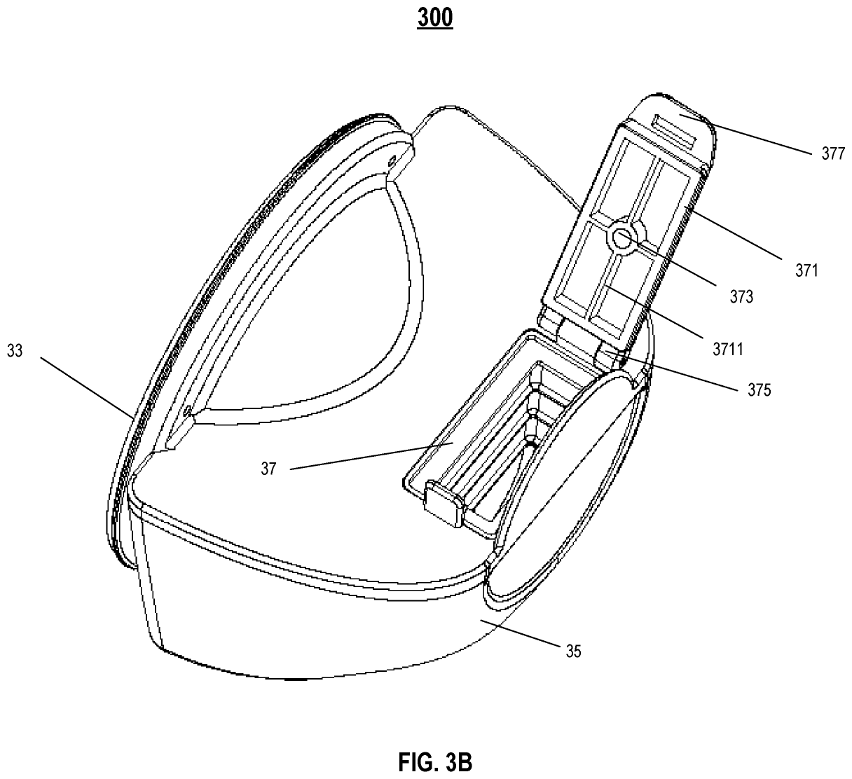

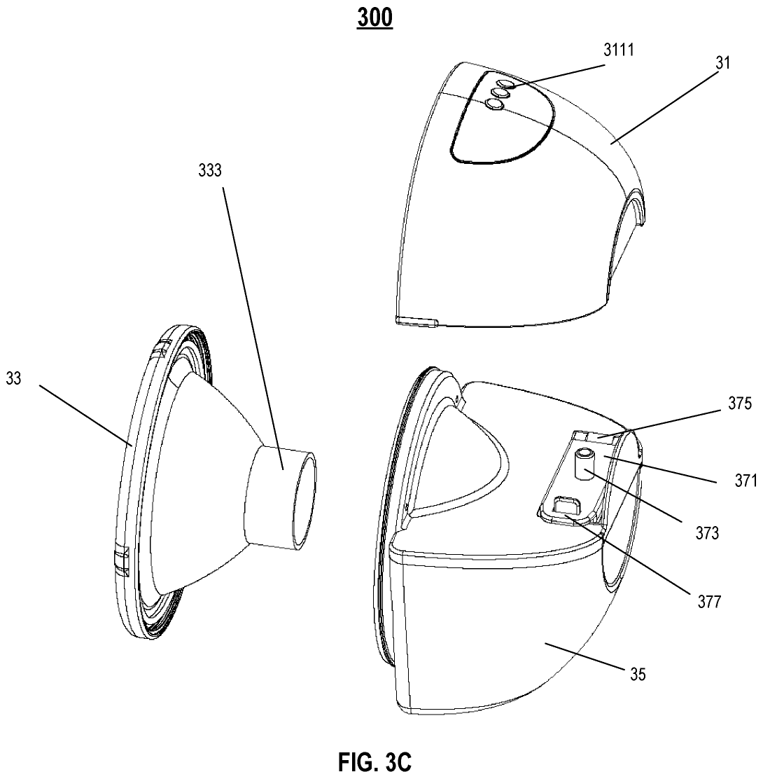

FIG. 3A illustrates a side view of an exemplary breast pump device 300, according to some aspects of the present disclosure. As shown in FIG. 3A, breast pump device 300 includes a main machine housing 31, as well as one or more buttons 3111 disposed on a top portion of main machine housing 31. Breast pump device 300 may be, at least in part, wearable or capable of being disposed inside a bra. Main machine housing 31 may further include a power port (not shown) that allows charging of a power source of the breast pump device or receiving of power provided from outside breast pump device 300 via the power port. Main machine housing 31 may accommodate a main machine of breast pump device 300, which includes several components of the main machine. The components may be the same as or similar to those of the implementations discussed in conjunction with breast pump devices 100 and 200 shown in FIGS. 1A to 2G.

Breast pump device 300 may further include a breast housing 33 that is detachably attached to main machine housing 31. In some implementations, breast housing 33 can be attached to machine housing 31 by, for example, fastening, buckling, locking, or fitting with each other. When attached together, breast pump device 300 may be leak-proof that prevents water, moisture, and air from entering itself. Breast housing 33 may be separated from main machine housing 31, so that one or both of them can be easily cleaned by user. Breast housing 33 may have a concave surface on a side opposite to main machine housing 31 to accommodate a breast of a nursing mother.

Breast pump device 300 may further include a milk container 35 that is detachably attached to at least one of main machine housing 31 or breast housing 33. In some implementations, milk container 25 can be attached to machine housing 21, breast housing 23, or both by, for example, fastening, buckling, locking, or fitting with each other. When attached together, breast pump device 200 can be leak-proof. When separated, milk container 25 can be easily cleaned by user. In some implementations, milk container 35 may be detachably attached to at least one of main machine housing 31 or breast housing 33 via other components that are coupled between milk container 35 and at least one of main machine housing 31 or breast housing 33. Milk container 35 is configured to collect milk from the breast of the nursing mother after expressing the milk.

FIG. 3B illustrates a top view of a milk container and a breast housing of exemplary breast pump device 300, according to some aspects of the present disclosure. As shown in FIG. 3B, breast pump device 300 may further include a suction diaphragm 37 detachably attached to milk container 35. In some implementations, milk container 35 may include a hole portion (not shown) in which suction diaphragm 37 can be inserted. Suction diaphragm 37 may include a duckbill valve (not shown) to guide the milk into the milk container 35.

FIG. 3C illustrates an exploded view of exemplary breast pump device 300, according to some aspects of the present disclosure. As shown in FIG. 3C, in some implementations, breast housing 33 may include a flange portion 333 that can be fit to a nipple of a breast to collect milk via a path connecting to breast housing 33. Flange portion 333 may be connected to a through path of a tee pipe structure (not shown). In some implementations, a cap 371 can be further coupled to a top portion of suction diaphragm 37. In some implementations, cap 371 is detachably attached to suction diaphragm 37. Cap 371 is also detachably attached to main machine housing 31. When the nipple of the breast is enclosed by breast housing 33 and aligned with flange portion 333, the pump in the main machine exhausts the air out of suction diaphragm 37 via cap 371 to generate negative pressure. As a result, the nipple enclosed by breast housing 33 and aligned with flange portion 333 of suction diaphragm 37 may express milk, which flows into milk container 35 via the duckbill valve.

As shown in FIGS. 3B and 3C, in some implementations, cap 371 includes a tube structure 373 that can be coupled to a suction hole (not shown) of the main machine. In some implementations, tube structure 373 may extend or coupled to another pipe so as to connect to the pump of main machine housing 31. In some implementations, tube structure 373 may be inserted into a recessed portion of the pump. In some implementations, cap 371 may further include a buckle 377 that can be used to fasten cap 371 to suction diaphragm 37, and a shaft 375 such that cap 371 can be opened to expose an inner surface of suction diaphragm 37 while still being attached to suction diaphragm 37.

In some implementations, cap 371 may further include a protrusion portion 3711 including at least one of concave or convex structures, strip structures, protrusion structures, formed on an inner surface of protrusion portion 3711 and surrounding an opening of tube structure 373. These structures formed on the inner surface of protrusion portion 3711 may retain some liquid (e.g., water, milk, etc.) or moisture inside of the breast pump machine (e.g., within suction diaphragm 37), thereby preventing the liquid or moisture from being sucked into the pump and thus damaging the pump.

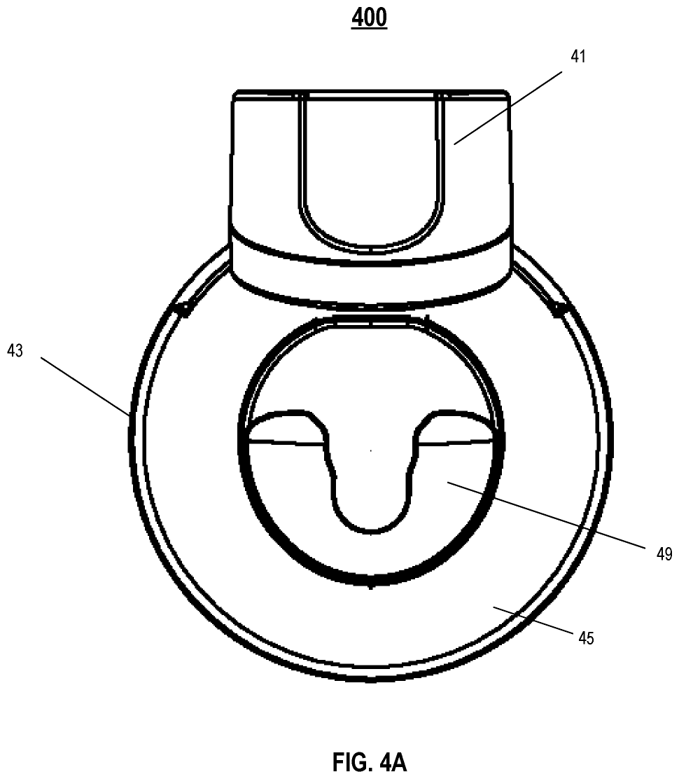

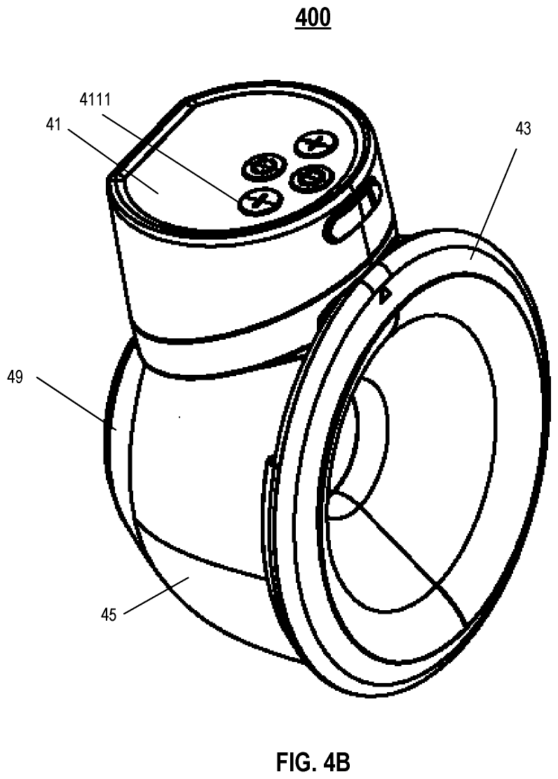

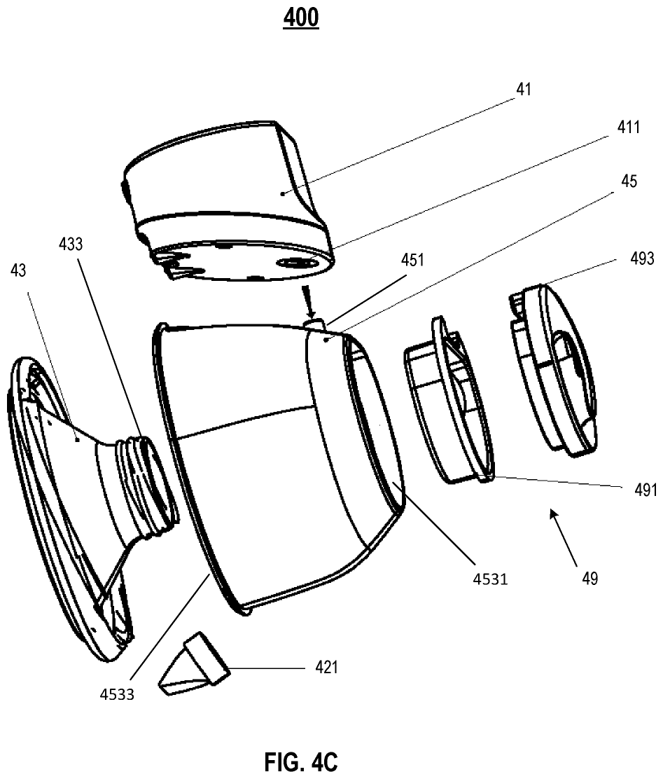

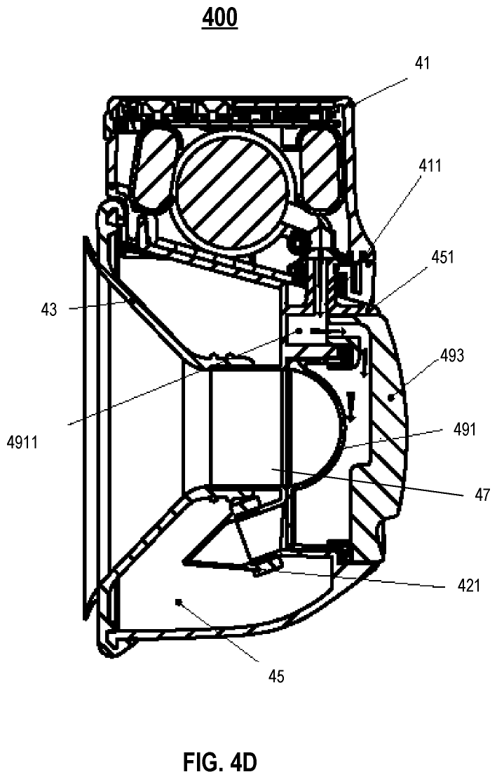

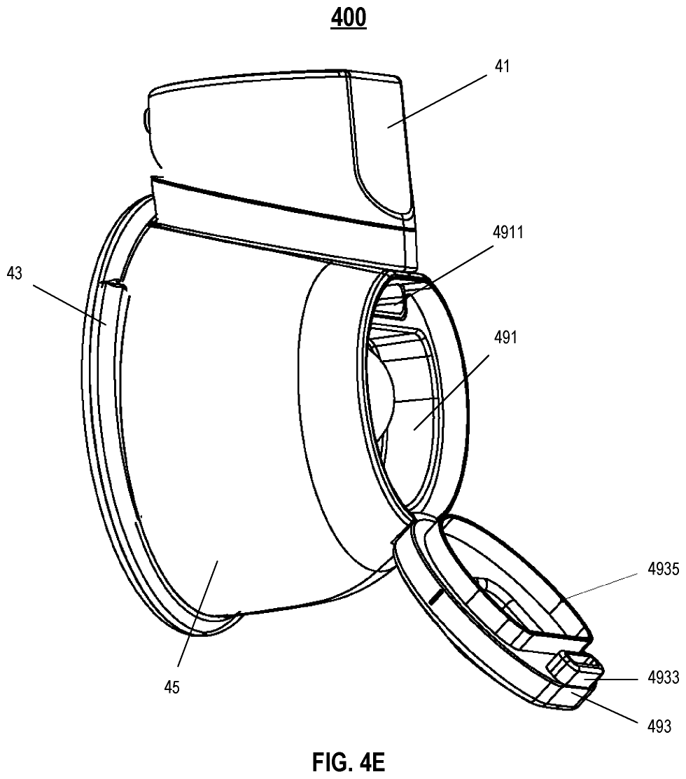

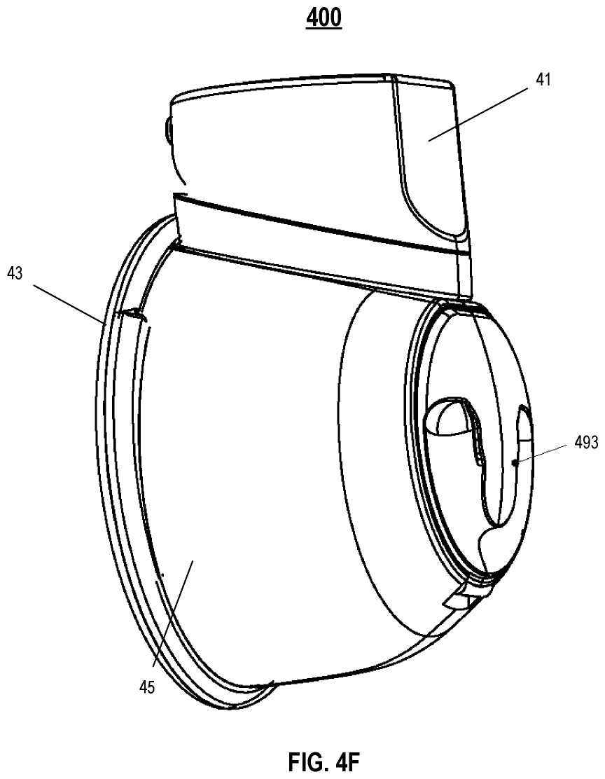

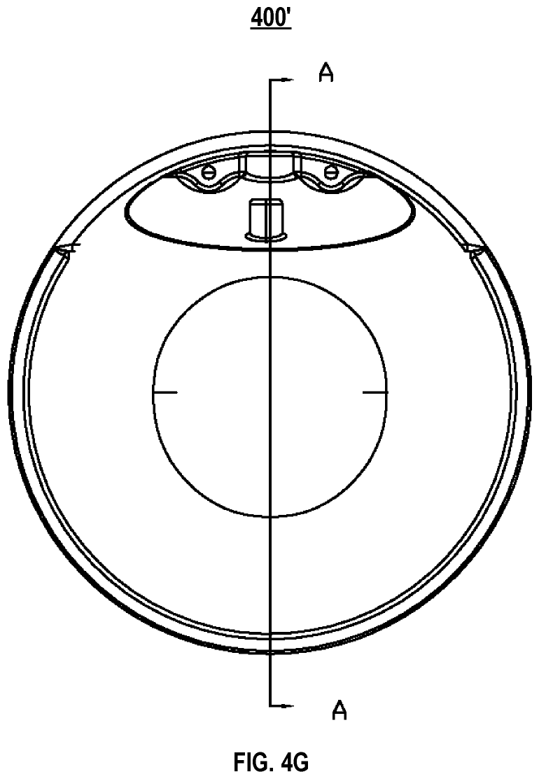

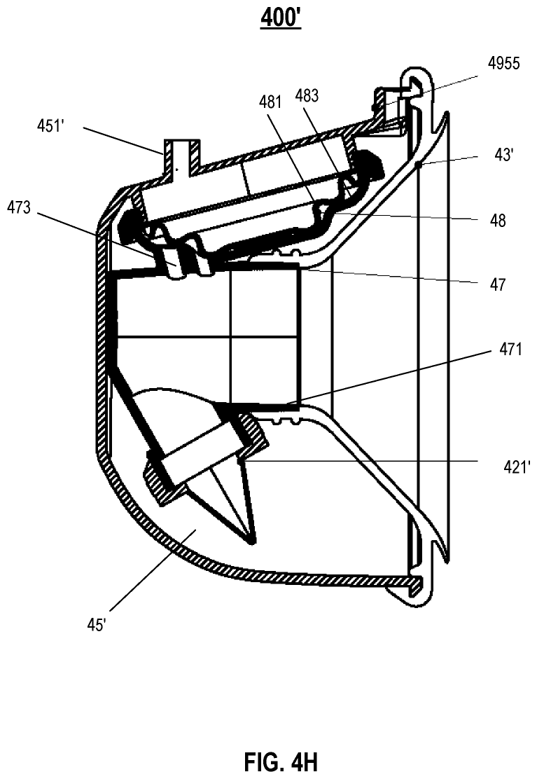

FIG. 4A illustrates a front view of an exemplary breast pump device 400, according to some aspects of the present disclosure. The main machine of breast pump device 400 and its housing is attached to the milk container via a hole (e.g., similar to tube structure 373 in FIG, 3C). Unlike breast pump device 300 in which the main machine and its housing are primarily operated inside the bra, the main machine and its housing of breast pump device 400 can be operated outside the bra (e.g., the main machine can be put in a pocket or on a desk), using a pipe or a hose to provide negative pressure to the tee pipe structure to express milk. As shown in FIG. 4A, breast pump device 400 includes a milk container 45, a suction chamber unit 49 coupled to a tee pipe structure (not shown) of milk container 45, a main machine housing 41 coupled to milk container 45, and a breast housing 43 coupled to milk container 45. Main machine housing 41, as mentioned above, can be detached from milk container 45. An additional pipe or hose (not shown) can be connected between main machine housing 41 and milk container 45.

FIG. 4B illustrates a rear view of exemplary breast pump device 400, according to some aspects of the present disclosure. As shown in FIG. 4B, breast pump device 400 further includes one or more buttons 4111 disposed on a top portion of main machine housing 41. In some implementations, buttons 4111 may include a power button, a switch button, or a control interface on which a user may start, stop, or change a pumping cycle, increase or decrease each peak pump pressure, or switch between different pre-programmed or pre-determined pressure profiles (e.g., mimicking a sucking pattern of a baby). Main machine housing 41 may further include a power port (not shown) that allows charging of a power source of the breast pump device or receiving of power provided from outside breast pump device 400 via the power port. Main machine housing 41 may accommodate the main machine of breast pump device 400, which includes several components of the main machine. The components may be the same as or similar to those of the implementations discussed in conjunction with breast pump devices 100, 200, and 300 shown in FIGS. 1A to 3C.

In some implementations, breast housing 43 is detachably attached to milk container 45. In some implementations, breast housing 43 can be attached to machine housing 41 by, for example, fastening, buckling, locking, or fitting with each other. When attached together, breast pump device 400 may be leak-proof that prevents water, moisture, and air from entering itself. Breast housing 43 may be separated from main machine housing 41, so that one or both of them can be easily cleaned by user. Breast housing 43 may have a concave surface on a side opposite to main machine housing 41 to accommodate a breast of a nursing mother. Main machine housing 41 is detachably attached to milk container 45 directly or via a pipe or a hose as mentioned above.

FIG. 4C illustrates an exploded view of exemplary breast pump device 400, according to some aspects of the present disclosure. As shown in FIG. 4C, in some implementations, breast housing 43 may include a flange portion 433 that can be fit to a nipple of a breast to collect milk via a path connecting to breast housing 43. Flange portion 433 may be connected to milk container 45 and tee pipe structure 47 from a first end 4533 of milk container 45. In some implementations, suction chamber unit 49 includes a suction chamber structure 491 coupled to milk container 45 via a second end 4531 of mile container 45. Suction chamber structure 491 may be attached directly to a duckbill valve 421 or coupled to duckbill valve 421 via a path in milk container 45. Suction chamber structure 491 may include a suction diaphragm (not shown). Duckbill valve 421 stays sealed when there is negative pressure being applied by the pump, thereby ensuring that the negative pressure is directly applied to milk container 45. Suction chamber unit 49 may include a suction cap 493 detachably attached to suction chamber structure 491. With suction cap 493 being attached, suction chamber unit 49 can be sealed airtight, such that the pump may generate the negative pressure upon the suction diaphragm of suction chamber structure 491, and the suction diaphragm may provide negative pressure to the nipple. In some implementations, main machine housing 41 may further include a suction hole 411 that can be coupled to a suction spout 451 on milk container 45. Suction hole 411 is connected to the pump in the main machine and provides negative pressure when the pump starts the pumping operation.

FIG. 4D illustrates a perspective view of exemplary breast pump device 400, according to some aspects of the present disclosure. As shown in FIG. 4D, suction hole 411 is coupled to suction spout 451. Suction spout 451 is connected to a filter chamber 4911 in which it may block liquid (e.g., milk or water) or moisture in milk container 45 or suction chamber structure 491 from leaking into the pump. When the pump starts the pumping operation, the negative pressure is generated in suction chamber structure 491 while duckbill valve 421 stays sealed, and the milk is then expressed to flow to the bottom of suction chamber structure 491. When the negative pressure is released, duckbill valve 421 is opened and the milk flows into milk container 45.

FIG. 4E illustrates a side view of exemplary breast pump device 400, according to some aspects of the present disclosure. As shown in FIG. 4E, filter chamber 4911 and a filter tube 4933 of suction cap 493 are relatively small in terms of the size as compared to suction chamber structure 491, and therefore with a filtering space provided between filter tube 4933 and filter chamber 4911 when they are connected, liquid or moisture can be prevented from being sucked into the pump. Suction cap 493 may further include a frame 4935 that holds suction chamber structure 491 in a fixed position when suction cap 493 is attached to milk container 45. FIG. 4F shows that when suction cap 493 is attached to milk container 45, suction chamber structure 491 can be secured between milk container 45 and suction cap 493.

FIG. 4G illustrates a top view of an exemplary breast pump device 400′, according to some aspects of the present disclosure, and FIG. 4H illustrates a cross-section view of breast pump device 400′ shown in FIG. 4G along an AA plane. It is noted that breast pump device 400′ is a variant of breast pump device 400 or breast pump device 100, and some of their components may be the same or similar. Also, those components not shown in the figures illustrating the implementations of breast pump device 400′ may be the same as those shown in the figures illustrating implementations of breast pump device 400 or breast pump device 100. For example, breast pump device 400′ may include a suction spout 451′ (corresponding to suction spout 451 in FIG. 4D) on milk container 45′ (corresponding to milk container 45 in FIG. 4D). In addition, breast pump device 400′ may include a main machine housing (corresponding to main machine housing 41 shown in FIG. 4D) coupled to suction spout 451′. However, suction chamber structure 491 and suction cap 493 in the implementations of breast pump device 400 as shown in FIG. 4D are replaced with a tee pipe structure 47 and a suction diameter unit 48 in the implementations of breast pump device 400′, as shown in FIG. 4H. It is also noted that the details of tee pipe structure 47 and suction diameter unit 48 are the same as or similar to those of tee pipe structure 17 and suction diameter unit 19 in the implementations of breast pump device 100, as shown in FIG. 1D. Thus, the details of tee pipe structure 47 and suction diameter unit 48 can be referred to those of tee pipe structure 17 and suction diameter unit 19, and thus will not be repeated here.

As shown in FIG. 4H, unlike the implementations of breast pump device 100 as shown in FIG. 1D, suction diameter unit 48 is embedded in milk container 45′ instead of the main machine housing. A buckle 4955 (similar to buckle 1955 in FIG. 1D) is located in a top portion of milk container 45′ so as to fasten suction diameter unit 48 to a fixed position. Like its counterpart of breast pump device 100, suction diameter unit 48 can be replaced or taken out for cleaning or washing. Suction diameter unit 48 may include a suction diaphragm 481 detachably attached to suction chamber 483. Breast pump device 400′ may further include a tee pipe structure 47 which includes a flange portion 471 that can be fit to a nipple of a breast to collect milk via a path connecting to breast housing 43′. An air path portion 473 is connected to suction diaphragm unit 48 to provide suction pressure, and a duckbill valve 421′ is connected to milk container 45′ to guide the milk into milk container 45′.

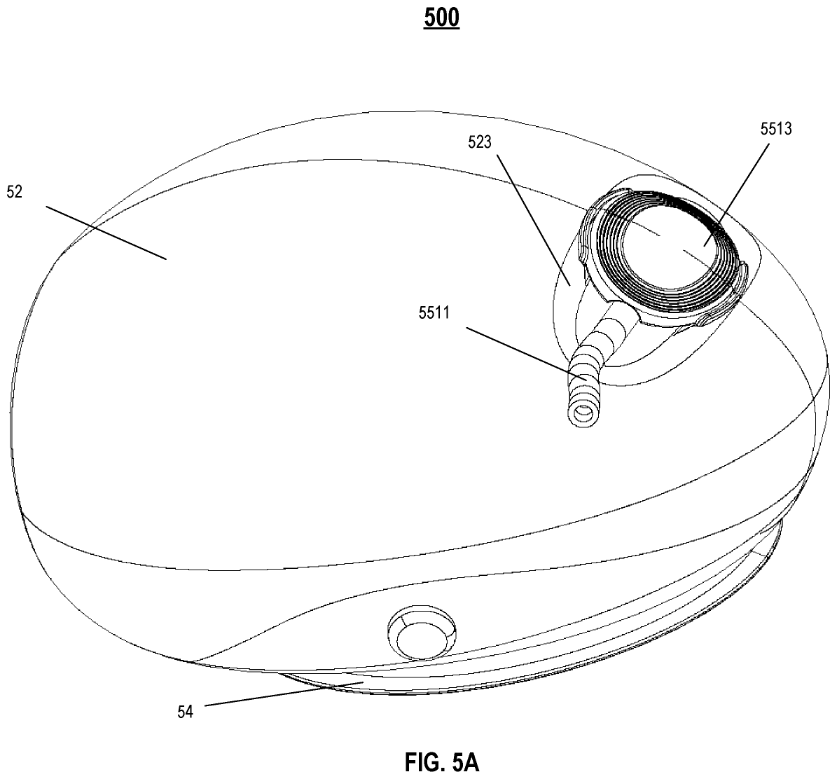

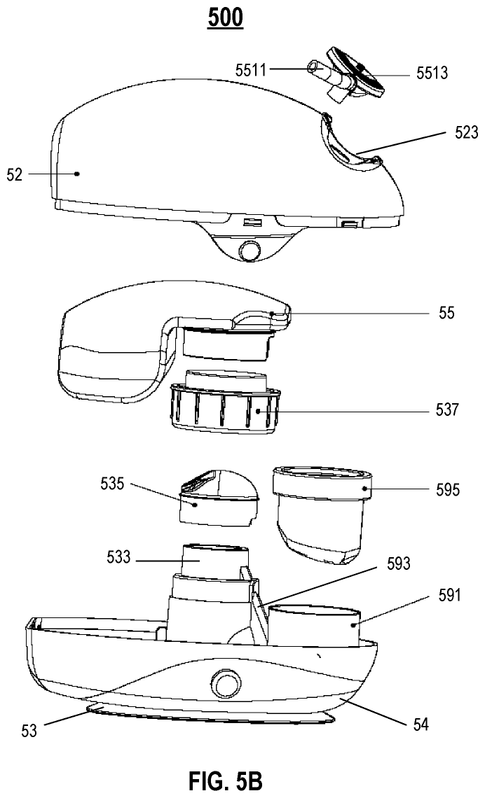

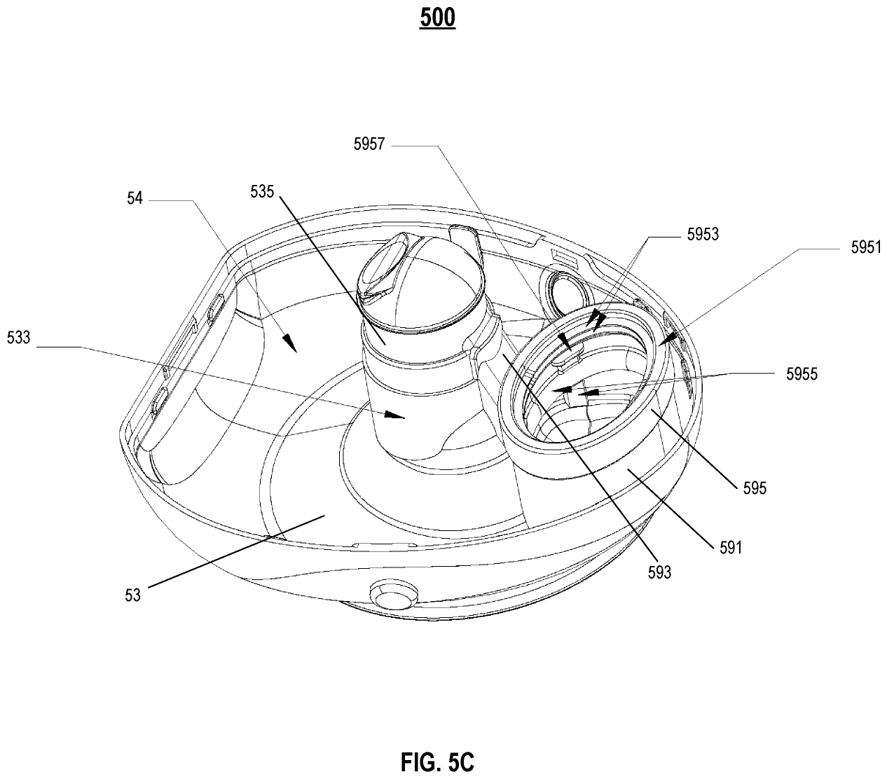

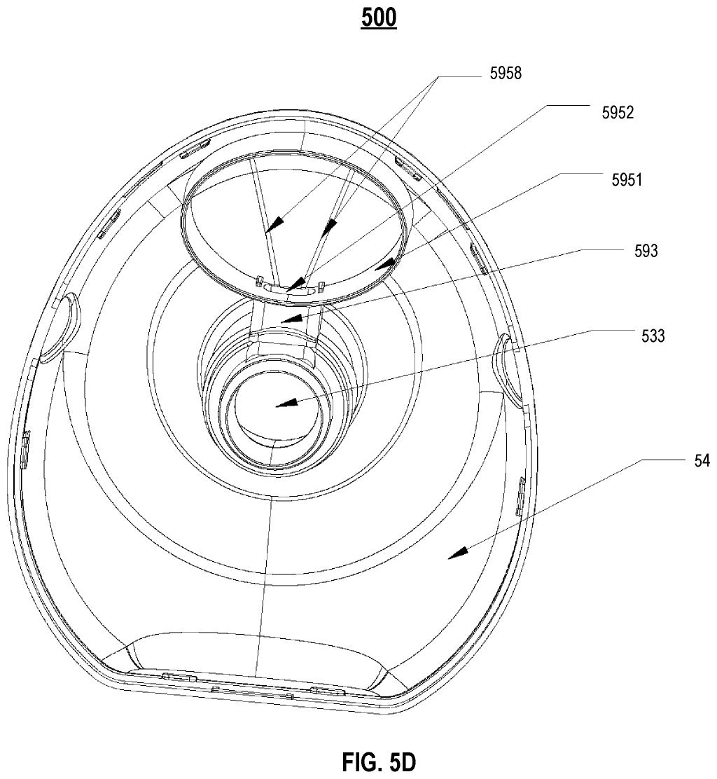

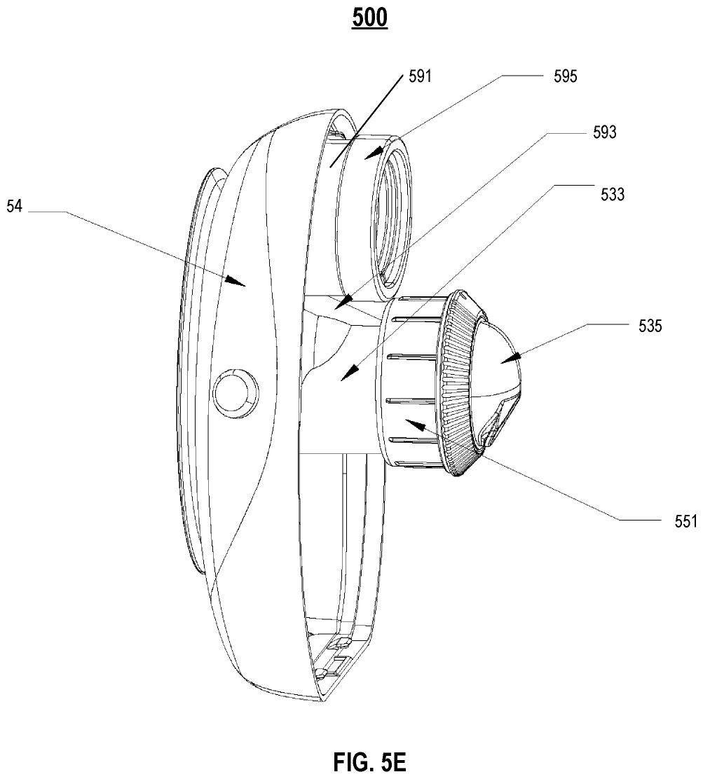

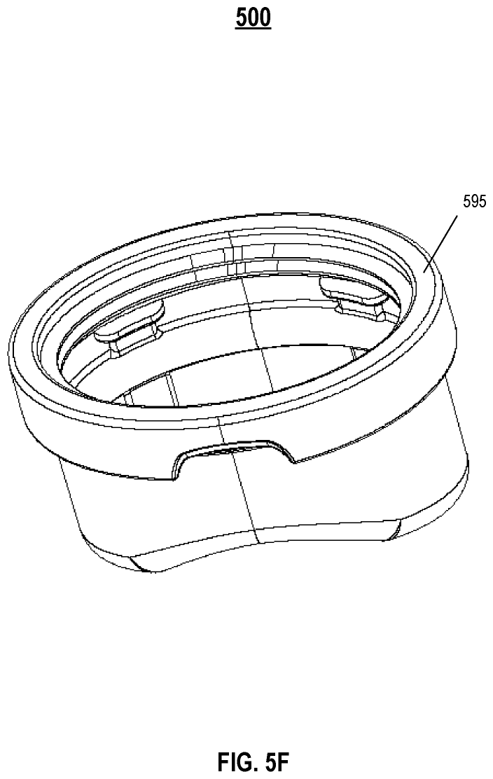

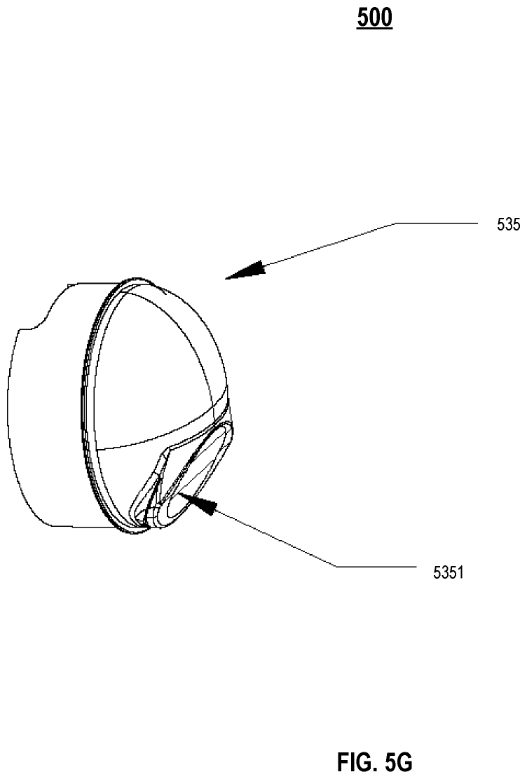

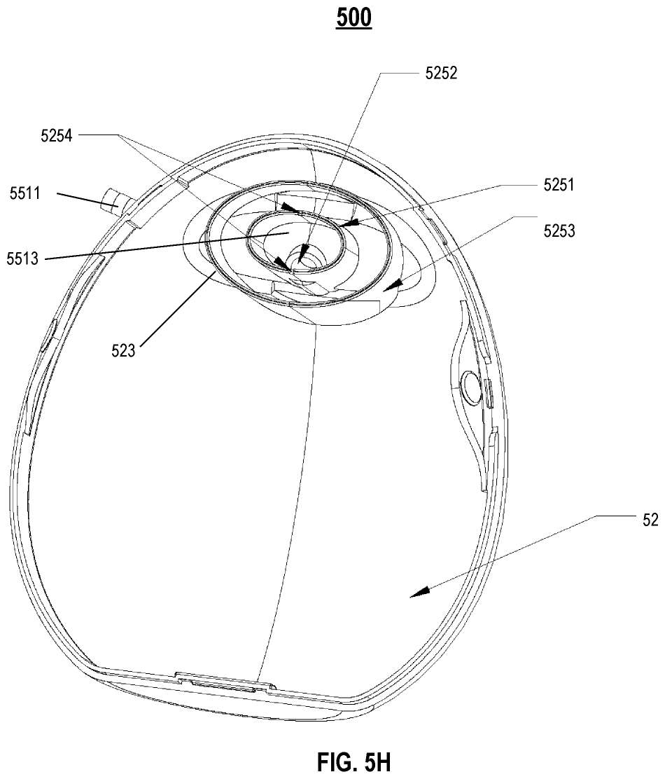

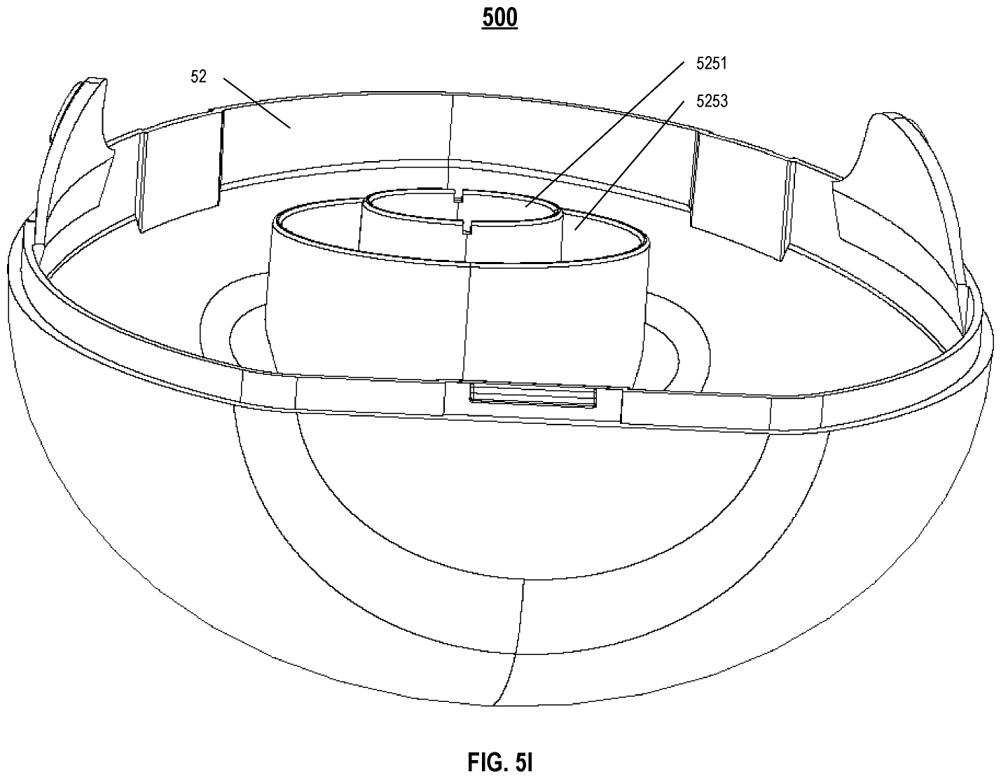

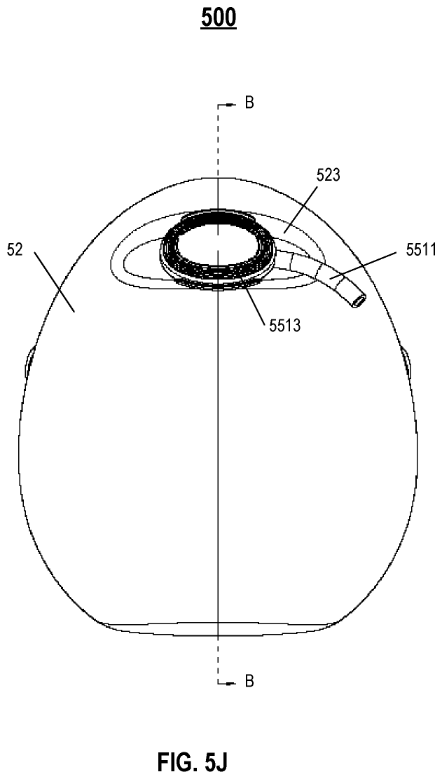

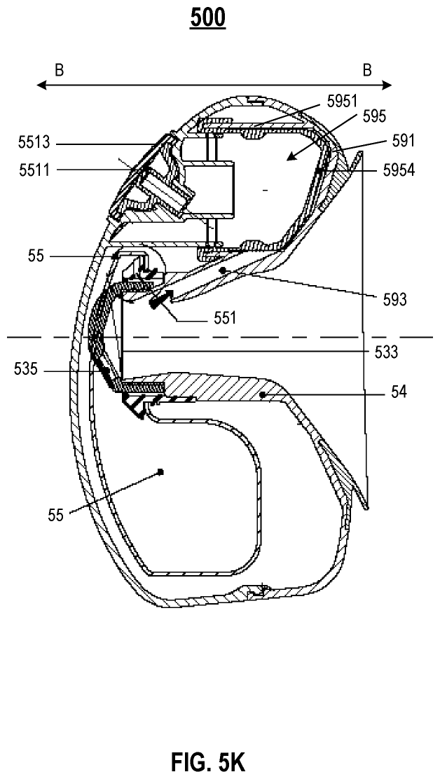

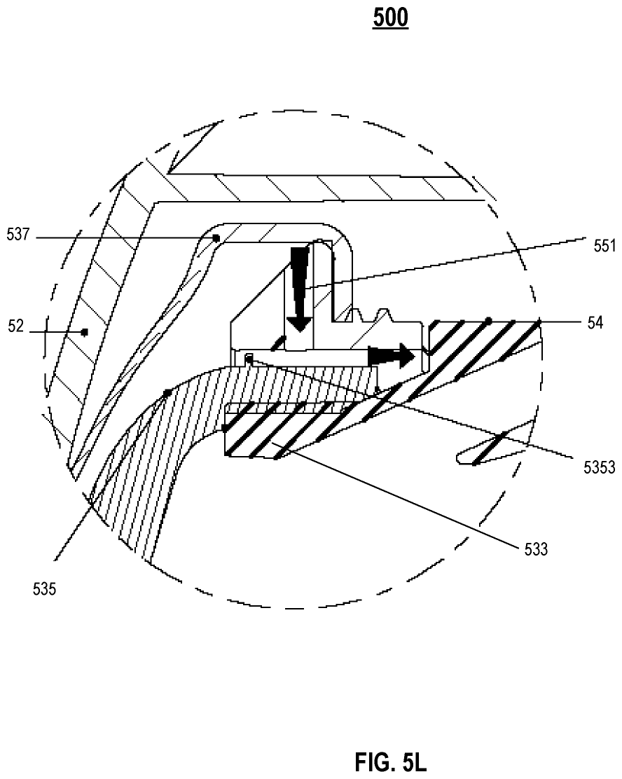

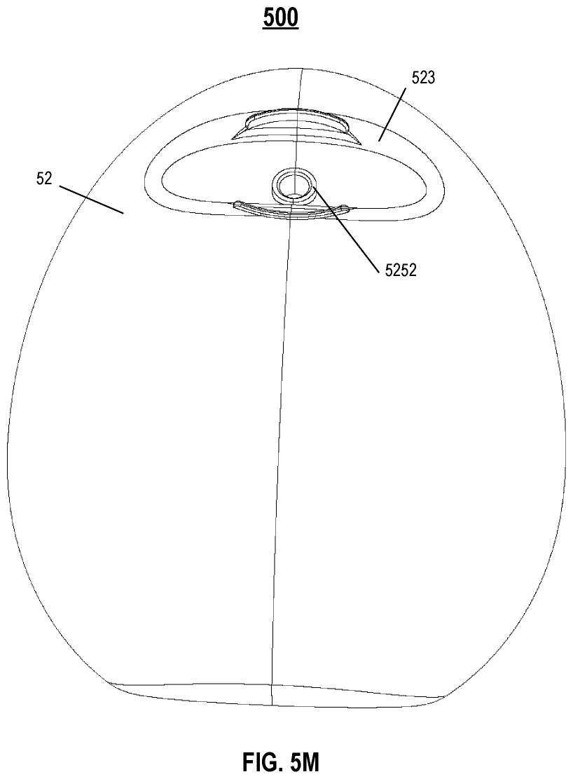

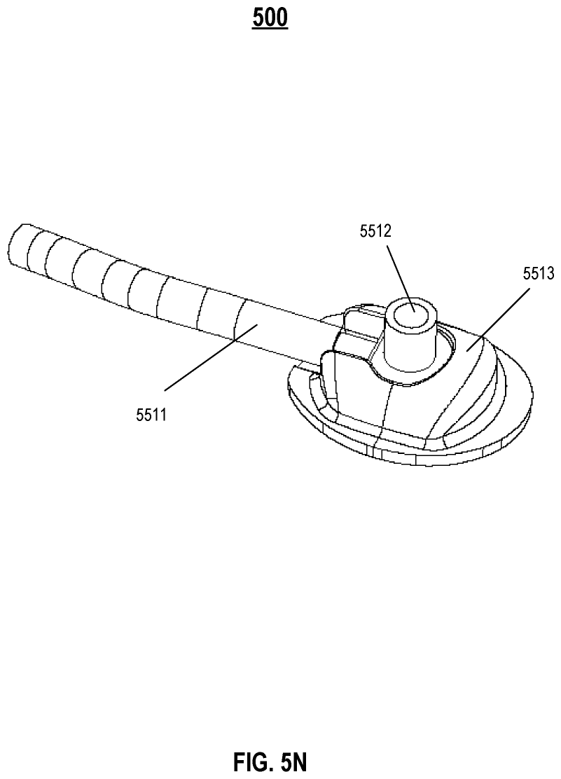

FIG. 5A illustrates a top view of an exemplary breast pump device 500, according to some aspects of the present disclosure. As shown in FIG. 5A, breast pump device 500 includes a first housing 52 and a second housing 54 coupled to first housing 52. A suction cap 5513 and a suction pipe 5511 are detachably attached to a socket 523 of first housing 52.

FIG. 5B illustrates an exploded view of exemplary breast pump device 500, according to some aspects of the present disclosure. As shown in FIG. 5B, in some implementations, breast pump device 500 may include a breast housing 53 coupled to a first side of second housing 54, and a milk container 55 coupled to first housing 52. Breast pump device 500 may include a flange portion 533 that can be fit to a nipple of a breast to collect milk via a path connecting to breast housing 53. Flange portion 533 may be connected to milk container 55 via a one-way valve 535 and a milk container cap 537. Each of one-way valve 535 and milk container cap 537 may include a thread structure or a locking mechanism so that they can be secured to flange portion 533 and milk container 55, respectively. In some implementations, one-way valve 535 may allow the milk to be released in one direction (i.e., from flange portion 533 into milk container 55), but not in the opposite direction. It is noted that one-way valve 535 is not limited to the one-way valve as illustrated, it can be any other types of valves as long as the valve can act as a backflow prevention valve or a one-way valve. It can also be a duckbill valve as disclosed in some implementations of the present application.

In some implementations, breast pump device 500 may further include a suction diaphragm unit 595 coupled to a second side of second housing 54 via a suction portion 591 of second housing 54. A suction channel 593 is provided between suction portion 591 and flange portion 533 so that the negative pressure can be applied to the nipple of the breast to express milk. In some implementations, suction diaphragm unit 595 is connected to suction cap 5513 and suction pipe 5511 via socket 523. For example, suction cap 5513 and suction pipe 5511 may be installed into suction diaphragm unit 595 via socket 523 directly or via an additional pipe (not shown) formed therebetween.

FIG. 5C illustrates a front view of second housing 54 of exemplary breast pump device 500, according to some aspects of the present disclosure. As shown in FIG. 5C, suction diaphragm unit 595 is coupled to suction portion 591. Suction diaphragm unit 595 may include a suction diaphragm frame 5951, and one or more seal portions 5953, both of which are configured to fasten a suction diaphragm (not shown) to a fixed position. A contact protrusion 5957 in suction diaphragm unit 595 is provided to contact and position the suction diaphragm. One or more rib structures 5955 are within suction diaphragm unit 595 to provide support of suction diaphragm unit 595 against negative pressure applied thereupon.

FIG. 5D illustrates another front view of second housing 54 of exemplary breast pump device 500, according to some aspects of the present disclosure. As shown in FIG. 5D, suction diaphragm unit 595 may further include a suction hole 5952 connected to suction channel 593. One or more exhaust grooves 5958 may also be provided to exhaust the air in the pressure chamber of suction diaphragm unit 595.

FIG. 5E further illustrates a side view of second housing 54 of exemplary breast pump device 500, according to some aspects of the present disclosure. As shown in FIG. 5E, suction channel 593 is between suction portion 591 and flange portion 533. One-way valve 535 is coupled to flange portion 533. A milk air vent 551 is provided and configured to provide air to be vented out so that the milk can flow into milk container 55 via one-way valve 535 smoothly (i.e., when air is vented out of the milk container, the milk can flow into the milk container without generating bubbles or spillage). FIG. 5F illustrates a side view of suction diaphragm unit 595 including a suction diaphragm, and FIG. 5G illustrates a side view of one-way valve 535 including a gate 5351 of one-way valve 535.

FIG. 5H illustrates a rear view of first housing 52 of exemplary breast pump device 500, according to some aspects of the present disclosure. As mentioned above, suction cap 5513 and suction pipe 5511 are coupled to suction diaphragm unit 595 via socket 523. Suction cap 5513 and suction pipe 5511 are installed via socket 523 from a first side of first housing 52. As shown in FIG. 5H, first housing 52 may include one or more diaphragm fixed parts (e.g., an inner diaphragm fixed part 5251 and an outer diaphragm fixed part 5253) in which suction cap 5513 and suction pipe 5511 are attached to each other via socket 523. These diaphragm fixed parts are configured to fasten and position the suction diaphragm from the middle to the periphery of the suction diaphragm. One or more exhaust guides 5254 may also be provided around inner diaphragm fixed part 5251 and outer diaphragm fixed part 5253 to release air or the negative pressure before or after installation of the suction diaphragm. In some implementations, suction cap 5513 may further include suction spout 5252 detachably attached to suction hole 5952. FIG. 5I illustrates a side view of first housing 52 to show that inner diaphragm fixed part 5251 protrudes further than outer diaphragm fixed part 5253 so that the suction diaphragm can be secured from the middle to the periphery of the suction diaphragm.

FIG. 5J further illustrates a front view of first housing 52 of exemplary breast pump device 500, according to some aspects of the present disclosure, and FIG. 5K illustrates a cross-section view of exemplary breast pump device 500 along a BB plane, according to some aspects of the present disclosure. As shown in FIG. 5K, a suction diaphragm 5954 is in an inner surface of suction diaphragm unit 595. When the nipple of the breast is inserted into flange portion 533 of breast housing 54, and the pump starts generating negative pressure via suction pipe 5511, suction diaphragm 5954 of suction diaphragm unit 595 starts to shrink and thus negative pressure starts applying to the nipple via suction channel 593 to express milk. The milk then flows into milk container 55 via one-way valve 535. In some implementations, as shown in FIG. 5K and the enlarged view in FIG. 5L, milk container 55 may further include a milk air vent 551 (which, for example, can be on milk container cap 538) configured to provide air to be vented out so that the milk can flow into milk container 55 via one-way valve 535 smoothly. In some implementations, one-way valve 535 may further include a sealing line 5353 between milk container cap 537 of milk container 55 and one-way valve 535 in order to prevent the milk being leaked out of milk container 55 via the interface between milk container 55 and one-way valve 535.

FIG. 5M further illustrates a front view of first housing 52 without a suction cap and a suction pipe of exemplary breast pump device 500, according to some aspects of the present disclosure, and FIG. 5N illustrates a front view of suction cap 5513 and suction pipe 5511 of exemplary breast pump device 500, according to some aspects of the present disclosure. In some implementations, as shown in FIGS. 5M and 5N, suction cap 5513 is connected to suction spout 5252 of socket 523 via pipe spout 5512. In some implementations, suction pipe 5511 may include stretchy or elastic material including flexible plastic, silicone, rubber, resin, or epoxy, or a combination of two or more of these materials.

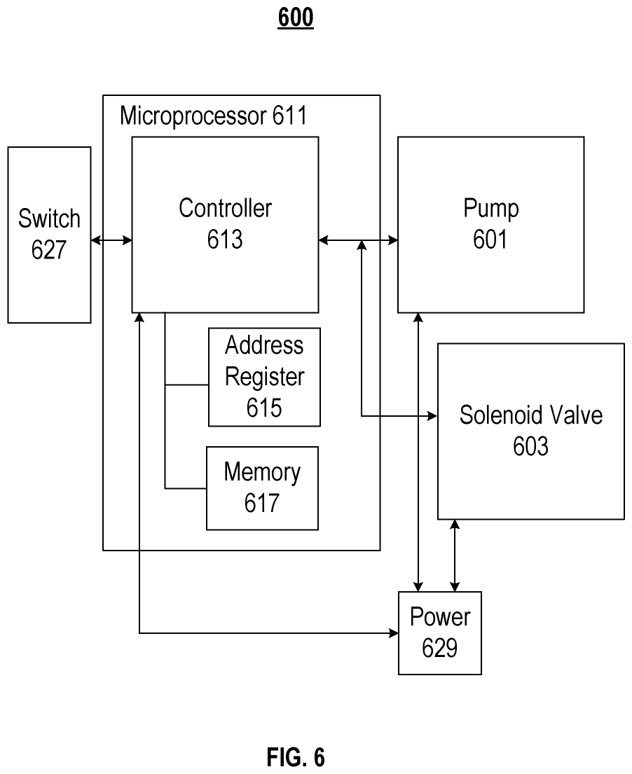

FIG. 6 illustrates a block diagram of an exemplary breast pump system 600, according to some aspects of the present disclosure. In some implementations, breast pump system 600 is at least partially embedded or coupled within the main machine according to implementations of the present application. For example, breast pump system 600 can be embedded within the main machine coupled to main machine housing 11, 21, 31, or 41. However, it is not limited to the implementation of the present disclosure, it can be any main machine coupled to or at least partially within the breast pump device, which can be any one of the breast pump devices 100, 200, 300, 400, 400′, or 500 as discussed above. As shown in FIG. 6, breast pump system 600 may include a microprocessor 611 having a controller 613, and an address register 615 and a memory 617 both coupled to controller 613. A pump 601 and a solenoid valve 603 are provided to generate negative pressure for any one of breast pump devices 100, 200, 300, 400, 400′, or 500, as described above. Microprocessor 611 is configured to control the operations of pump 601 and solenoid valve 603 in order to perform all the functions of the pump and the solenoid valve including the functions to start, stop, or change a pumping cycle, to increase or decrease each peak pump pressure, or to switch between different pre-programmed or pre-determined pressure profiles (e.g., mimicking a sucking pattern of a baby).

The foregoing description of the specific implementations will so reveal the general nature of the present disclosure that others can, by applying knowledge within the skill of the art, readily modify and/or adapt for various applications of such specific implementations, without undue experimentation, and without departing from the general concept of the present disclosure. Therefore, such adaptations and modifications are intended to be within the meaning and range of equivalents of the disclosed implementations, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

Implementations of the present disclosure have been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The Summary and Abstract sections may set forth one or more but not all exemplary implementations of the present disclosure as contemplated by the inventor(s), and thus, are not intended to limit the present disclosure and the appended claims in any way.

The breadth and scope of the present disclosure should not be limited by any of the above-described exemplary implementations, but should be defined only in accordance with the following claims and their equivalents.

1. A breast pump device, configured as an at least in part in-bra wearable device, wherein the breast pump device comprises:
a main machine housing;
an electric pump configured to provide a first negative pressure and coupled to the main machine housing;
a control circuit coupled to the electric pump and configured to control operations of the electric pump;
a breast housing configured to accommodate a breast of a user;
a milk container;
a tee pipe structure coupled to the breast housing or the milk container; and
a suction diaphragm unit coupled between the main machine housing and the tee pipe structure, wherein the suction diaphragm unit is configured to generate a second negative pressure to the breast of the user in response to the first negative pressure via the tee pipe structure while preventing liquid or moisture from entering the electric pump, and
wherein the suction diaphragm unit comprises a diaphragm cover, the diaphragm cover comprises a tube structure connected to a suction hole of the main machine housing to transfer the first negative pressure.

2. The breast pump device of claim 1, wherein the suction diaphragm unit comprises:
a suction chamber; and
a suction diaphragm detachably attached to the suction chamber,
wherein the diaphragm cover detachably attached to the suction chamber and configured to secure the suction diaphragm between the diaphragm cover and the suction chamber.

3. The breast pump device of claim 2, wherein the diaphragm cover further comprises:
a protrusion portion comprising at least one of concave or convex structures, strip structures, or protrusion structures, formed on an inner surface of the protrusion portion and surrounding an opening of the tube structure.

4. The breast pump device of claim 2, wherein the suction chamber comprises at least one material selected from Acrylonitrile Butadiene Styrene (ABS) plastic, polypropylene, polystyrene, nylon, polycarbonate, or methacrylate, and wherein the suction diaphragm comprises at least one material selected from silicone, rubber, resin, or epoxy.

5. (canceled)

6. The breast pump device of claim 2, wherein a material of the suction chamber is stiffer than that of the suction diaphragm.

7. The breast pump device of claim 2, wherein the suction diaphragm is configured to fit over a rim of the suction chamber to seal the suction chamber airtight, and wherein the diaphragm cover comprises a buckle detachably attached to the suction chamber.

8. The breast pump device of claim 2, further comprising:
a shaft, wherein the suction chamber, the suction diaphragm, and the diaphragm cover are connected to the shaft.

9. The breast pump device of claim 1, wherein the breast housing comprises:
a flange portion coupled to the tee pipe structure and configured to be coupled to a nipple of the breast; and
a concave surface on a side of the breast housing opposite to the main machine housing to accommodate the breast.

10. The breast pump device of claim 1, wherein the tee pipe structure comprises:
a thread structure coupled to the milk container and configured to fasten the tee pipe structure to the milk container; and
a duckbill valve coupled to the milk container and configured to guide the milk into the milk container.

11. A breast pump device, configured as an at least in part in-bra wearable device, wherein the breast pump device comprises:
a main machine housing;
an electric pump configured to provide a first negative pressure and coupled to the main machine housing;
a control circuit coupled to the electric pump and configured to control operations of the electric pump;
a breast housing configured to accommodate a breast of a user;
a milk container;
a suction diaphragm coupled to the milk container or the breast housing and configured to apply a second negative pressure to the breast of a nursing mother in response to the first negative pressure; and
a filtering cap coupled between the suction diaphragm and the main machine housing,
wherein the filtering cap comprises:
a first lid;
a cap body coupled to the first lid; and
a structure coupled to the cap body and configured to retain liquid or moisture between the cap body and the first lid.

12. The breast pump device of claim 11, wherein the filtering cap comprises:
one or more cap openings configured to block or reduce liquid or moisture from passing through while allowing air to pass through, wherein the one or more cap openings are arranged in one or more arrays.

13. The breast pump device of claim 12, wherein none of the one or more cap openings is aligned with a suction hole of the main machine housing, and
wherein the suction hole is connected to the electric pump.

14. The breast pump device of claim 11, wherein the first lid comprises a lid opening, the cap body comprises a cap hole, and the lid opening is aligned with a suction hole of the main machine housing and the cap hole of the cap body.

15. The breast pump device of claim 14, wherein the structure comprises at least one of a concave structure or a convex structure around the cap hole.

16. A breast pump device, configured as an at least in part in-bra wearable device, wherein the breast pump device comprises:
a main machine housing;
an electric pump configured to provide a first negative pressure and coupled to the main machine housing;
a control circuit coupled to the electric pump and configured to control operations of the electric pump;
a breast housing configured to accommodate a breast of a user;
a milk bowl;
a suction diaphragm detachably coupled to the breast housing or the milk bowl, and configured to generate a second negative pressure to the breast of the user in response to the first negative pressure; and
a cap coupled to the suction diaphragm or the milk bowl,
wherein the cap comprises a tube structure configured to be plugged in to a suction hole of the main machine housing to apply the first negative pressure to the suction diaphragm.

17. The breast pump device of claim 16, wherein the cap comprises:
a buckle configured to fasten the cap to the suction diaphragm.

18. The breast pump device of claim 16, further comprises: a shaft, wherein the cap is attached to the suction diaphragm via the shaft.

19. The breast pump device of claim 16, wherein the suction hole is in a recessed portion of the main machine housing, and the tube structure is inserted into the recessed portion of the main machine housing.

20. The breast pump device of claim 16, wherein the cap further comprises: a protrusion portion comprising at least one of concave or convex structures, strip structures, or protrusion structures, formed on an inner surface of the protrusion portion and surrounding the suction hole.
